(19) United States
(12) Patent Application Publication
(10) Pub. No.: US 2024/0369490 A1
(43) Pub. Date: Nov. 7, 2024

(54) CONFIGURABLE HANDHELD BIOLOGICAL ANALYZERS FOR IDENTIFICATION OF BIOLOGICAL PRODUCTS BASED ON RAMAN SPECTROSCOPY USING ENSEMBLE ARTIFICIAL INTELLIGENCE

(71) Applicant: AMGEN INC., Thousand Oaks, CA (US)

(72) Inventors: Robert Joseph Soto, Thousand Oaks, CA (US); David Meriage, Thousand Oaks, CA (US)

(21) Appl. No.: 18/650,410

(22) Filed: Apr. 30, 2024

Related U.S. Application Data
(60) Provisional application No. 63/463,187, filed on May 1, 2023.

(51) Int. Cl. G01N 21/65; G06N 20/20

(52) U.S. Cl. CPC G01N 21/65; G06N 20/20; G01N 2201/126

(57) ABSTRACT

Configurable handheld biological analyzers and related biological analytics methods are described for identification of biological products based on Raman spectroscopy using ensemble artificial intelligence (AI). A biological classification ensemble model configuration is loaded into a computer memory of a configurable handheld biological analyzer having a processor and a scanner. The biological ensemble classification model configuration includes a biological classification ensemble model having an unsupervised model and a supervised model. The biological classification ensemble model configured to receive a Raman-based spectra dataset defining a biological product sample as scanned by the scanner. A spectral preprocessing algorithm is executed to reduce a spectral variance of the Raman-based spectra dataset. The biological ensemble classification model identifies a biological product type based on the first Raman-based spectra dataset.

### RELATED APPLICATION

This application claims the benefit of U.S. Provisional Application No. 63/463,187 (filed on May 1, 2023), which is incorporated by reference herein in its entirety.

### FIELD OF DISCLOSURE

The present disclosure generally relates to configurable handheld biological analyzers, and, more particularly, to systems and methods for using configurable handheld biological analyzers to identify or classify biological products based on Raman spectroscopy using ensemble artificial intelligence (AI).

### BACKGROUND

Development and manufacture of pharmaceutical and biotechnology products generally requires the measurement or identification of raw materials used to develop such products. The purpose of identification testing of products is to provide assurance of product identity. Situations that require identification testing include distribution of product to clinical sites, import testing, and transfer between network sites. In addition, measurement or identification of biological products can be important to ensure the quality of a development or manufacturing process, and ultimately the quality of the finished products themselves, for the purpose of meeting quality standards and/or regulatory requirements.

The use of Raman spectroscopy for measurement and identification of biological products is a relatively new concept. Generally, Raman spectroscopy can be used to probe a chemical or biological structure of a raw material or product. Raman spectroscopy is a non-destructive chemical or biological analysis technique that measures the interaction of light with a product or material, such as the interaction of light with biological attributes or chemical bonds of a product or material. Raman spectroscopy provides a light scattering technique where a molecule of a sample material or product scatters incident light from a high intensity laser light source. Typically, most of the scattered light is at the same wavelength (color) as the laser source and does not provide useful information—this is called Rayleigh scatter. However, a small amount of light is scattered at different wavelengths (colors), which is caused by the chemical or molecular structure of the material or product being analyzed—this is called Raman scatter and may be analyzed or scanned to generate Raman-based data of the material or product being analyzed.

Analysis of Raman scatter can yield detailed information regarding the characteristics of a material or product, including its chemical structure and/or identity, contamination and impurity, phase, crystallinity, intrinsic stress/strain, and/or molecular interactions, etc. Such detailed information can be present in the Raman spectrum of a material. A Raman spectrum can be visualized to show a number of peaks across various light wavelengths. The Raman spectrum can show the intensity and wavelength position of the Raman scattered light. Each peak can correspond to a specific molecular bond vibration associated with the material or product being analyzed.

Typically, a Raman spectrum provides a distinct chemical or biological “fingerprint” for a particular material, molecule, or product, and can be used to verify the identity of the particular material, molecule, or product—and/or distinguish it from others. In addition, Raman spectral libraries—compilations of Raman spectra, typically for many different materials—are often used for identification of a material based on its Raman spectrum. That is, Raman spectral libraries can be searched to find a match having a Raman spectrum for a given material or product being measured, to thereby identity the given product material or product.

Analyzers implementing Raman spectroscopy currently exist for identifying raw materials and products. For example, Thermo Fisher Scientific Inc. provides a Raman-based handheld analyzer identifiable as the TruScan™ RM Handheld Raman Analyzer. However, the use of such existing scanners can be problematic for use with materials and/or products having similar Raman spectra, such as pharmaceutical and biotechnology materials or products having similar Raman spectra. For example, variance among Raman spectra of similar products may cause an existing Raman-based handheld analyzer to incorrectly identify, e.g., by outputting a Type 1 Error (false positive) or Type II error (false negative) for a pharmaceutical or biotechnology product. A major source of variance or error originates from differences among the Raman-based analyzers, including differences such as variability in any of the software, manufacture, age, component(s), operating environment (e.g., temperature), or other such differences of the Raman-based analyzers.

Known approaches typically fail to address the error caused by the variance or variability among handheld analyzers. For example, in one known approach, data from several analyzers may be used to develop a static mathematical equation for use across several analyzers. Generally, however, the difficulty with this approach is that instrument performance may vary over time. Many times, it is also impractical or impossible to have routine access to all of these instruments. In particular, the data for construction of the static mathematical equation is generally not available, especially for new analyzers, where a manufacturer may not provide new specifications for new analyzers in advance. This prevents the development and maintenance of the static mathematical equation, especially as such new analyzers are developed over time, and given that the development of a static mathematical equation typically requires a large number of samples for different analyzer to be accurate. Moreover, without such new specifications for new analyzers, the static mathematical equation may not be compatible when executing the static mathematical equation on new analyzers. In addition, differences in the manufacturing or quality control of analyzers, especially among different manufacturers, for example, causes the static mathematical equation to become over tolerant as to variability, thereby creating a static mathematical equation that itself that is too variable for accurate measurement and/or identification of biological products.

In a second known approach, the data from a given analyzer is standardized, where a child-to-parent instrument map is created for a given group of analyzers. This approach, however, is limited because construction of a child-to-parent instrument map generally requires data from both parent and child instruments, which is typically difficult and/or computationally costly to implement or maintain, especially over longer periods of time as new generations of analyzers are developed, thereby requiring numerous permutations and types of child-to-parent instrument maps. In addition, with respect to the biopharmaceutical industry, user access to the child instruments is restricted, which also limits the child-to-parent instrument map approach. Furthermore, biopharmaceutical manufacturing is subject to regulations, for example requirements for GMP environments, which may require revalidations to a child-to-parent transfer map. Such revalidations can consume substantial time and resources.

In a third known approach, data from a given analyzer is also standardized, but where the variability among analyzers is ignored or treated as trivial. Such an approach is not, however, desirable given that analyzer-to-analyzer variability typically impacts accurate identification and measurement of raw material and/or biological products, and should, therefore be taken into account.

In yet a fourth approach, a trained model may be used for identification of biological products based on Raman spectroscopy. This approach is described by publication WO 2021/081263 titled “Configurable Handheld Biological Analyzers for Identification of Biological Products based on Raman Spectroscopy,” filed as PCT/US2020/056961 on Oct. 23, 2020.

For the foregoing reasons, there is a need for systems and methods for using configurable handheld biological analyzers to identify or classify biological products based on Raman spectroscopy using ensemble artificial intelligence (AI), which are configured to reduce variability, and increase compatibility, among similarly configured, configurable handheld biological analyzers when compared to known solutions.

### SUMMARY

The disclosure of the present application describes use of Raman spectroscopy, via handheld analyzer(s), for identification of biological products. Moreover, the disclosure of the present specification describes the use of configurable handheld biological analyzers, systems, and methods to overcome limitations generally associated with known methods of using Raman spectra to measure biological products. For example, the Raman spectra among certain biological products can be too similar to distinguish with known methods of using Raman spectra, which typically depend on generalized statistical algorithms. Raman spectra measurements can be especially problematic when instrument-to-instrument variability is introduced, causing, for example, Type I and Type II errors among the various analyzers. As described herein, such variability can be caused by any one or more of differences in software, manufacture, age, components, operating environment (e.g., temperature), or other differences of Raman-based analyzers. This problem manifests itself especially during the development or manufacturer of biological products, because analyzer-to-analyzer variability can be key factor affecting quality, robustness, and/or transferability in a manufacturing or development process related to a pharmaceutical or biological product. Accordingly, in various embodiments disclosed herein, configurable handheld biological analyzers are described, for example, that use configurations that use specific preprocessing algorithms and/or multivariate data analysis to (1) ensure that measurement and/or identification of materials or products is sensitive and/or specific, and (2) ensure the compatibility and configuration, as developed on a first set of analyzers, is transferable and/or implementable to additional analyzers, such as new analyzers within a “network” or group of analyzers.

Accordingly, in various embodiments herein, a configurable handheld biological analyzer for identification of biological products based on Raman spectroscopy using ensemble artificial intelligence (AI) is disclosed. The configurable handheld biological analyzer may comprise a first housing adapted for handheld manipulation and a first scanner carried by the first housing. The configurable handheld biological analyzer may further comprise a first processor communicatively coupled to the first scanner. The configurable handheld biological analyzer may further comprise a first computer memory communicatively coupled to the first processor. In various aspects, the first computer memory may be configured to load a biological ensemble classification model configuration. The biological ensemble classification model configuration may comprise a biological classification ensemble model comprising an unsupervised model and a supervised model. The unsupervised model may be trained with Raman-based spectra training data to configure the unsupervised model to output a first indicator of one or more biological product types. The supervised model may be trained with Raman-based spectra training data to configure the supervised model to output a second indicator of the one or more biological product types. Still further, the biological classification ensemble model configuration may comprise one or more spectral preprocessing algorithms. The first processor may be configured to execute the one or more spectral preprocessing algorithms to reduce a spectral variance of a first Raman-based spectra dataset when the first Raman-based spectra dataset is received by the first processor. The biological classification ensemble model may further be configured to execute on the first processor, where the first processor is configured to (1) receive a first Raman-based spectra dataset defining a first biological product sample as scanned by the first scanner, and (2) identify, with the biological classification ensemble model, a biological product type of the one or more biological product types based on the first Raman-based spectra dataset.

In additional embodiments disclosed herein, a biological analytics method for identification of biological products based on Raman spectroscopy using ensemble artificial intelligence (AI) is disclosed. The biological analytics method may include loading, into a first computer memory of a first configurable handheld biological analyzer having a first processor and a first scanner, a biological ensemble classification model configuration. The biological ensemble classification model configuration may comprise a biological classification ensemble model comprising an unsupervised model and a supervised model. The unsupervised model may be trained with Raman-based spectra training data to configure the unsupervised model to output a first indicator of one or more biological product types. Further, the supervised model may be trained with Raman-based spectra training data to configure the supervised model to output a second indicator of the one or more biological product types. The biological analytics method may further include receiving, at the first processor, a first Raman-based spectra dataset defining a first biological product sample as scanned by the first scanner. The biological analytics method may further include executing, by the first processor, one or more spectral preprocessing algorithms as specified by the biological ensemble classification model configuration, to reduce a spectral variance of the first Raman-based spectra dataset. The biological analytics method may further include identifying, with the biological classification ensemble model, a biological product type based on the first Raman-based spectra dataset.

In still further additional embodiments disclosed herein, tangible, non-transitory computer-readable medium (e.g., a computer memory) storing instructions for identification of biological products based on Raman spectroscopy using ensemble artificial intelligence (AI) is described. The instructions, when executed by one or more processors of a configurable handheld biological analyzer, may cause the one or more processors of the configurable handheld biological analyzer to load, into a first computer memory of a first configurable handheld biological analyzer having a first processor and a first scanner, a biological ensemble classification model configuration. The biological ensemble classification model configuration may comprise a biological classification ensemble model comprising an unsupervised model and a supervised model. The unsupervised model may be trained with Raman-based spectra training data to configure the unsupervised model to output a first indicator of one or more biological product types. Further, the supervised model may be trained with Raman-based spectra training data to configure the supervised model to output a second indicator of the one or more biological product types. The instructions, when executed, may further cause the one or more processors to receive, at the first processor, a first Raman-based spectra dataset defining a first biological product sample as scanned by the first scanner. The instructions, when executed, may further cause the one or more processors to execute, by the first processor, one or more spectral preprocessing algorithms as specified by the biological ensemble classification model configuration, to reduce a spectral variance of the first Raman-based spectra dataset. The instructions, when executed, may further cause the one or more processors to identify, with the biological classification ensemble model, a biological product type based on the first Raman-based spectra dataset.

Benefits of the present application include development of biological ensemble classification model(s) (e.g., multivariate analysis model(s)) that yield consistent results for a same pharmaceutical or biological product (e.g., therapeutic products/drugs) across different analyzers, including different analyzers used to scan Raman-based datasets used to construct the biological ensemble classification model. As described herein, multiple analyzers, or data multiple sets of Raman spectra generated by such analyzers, may be used to construct the biological ensemble classification model.

Further, as described herein, the biological ensemble classification models are configurable and transferable among configurable handheld biological analyzers and may comprise Raman spectral preprocessing, ensemble model chaining, and discriminating statistical analysis to reduce variability among configurable handheld biological analyzers. For example, use of the biological ensemble classification model, as described herein, improves over existing analyzers because it reduces variability among instruments/analyzers, requires no data from child instruments to develop, and may be used across different analyzers implementing different software, having different software or software versions, having different manufactures, ages, operating environments (e.g., temperatures), hardware, or other such differences.

Moreover, a biological ensemble classification model's accuracy may be increased by applying preprocessing techniques (e.g., spectral preprocessing algorithms, as described herein) to minimize statistical Type I and/or Type II error of the biological ensemble classification model's output, and, therefore improve the output the configurable handheld biological analyzer(s), on which the biological ensemble classification model is installed/configured.

In addition, in some embodiments, configurable handheld biological analyzer(s) may use a biological ensemble classification model to distinguish biological products/drugs having similar protein structure, protein concentration, and/or formulations. This provides a flexible approach, as biological ensemble classification models may be generated with various, different, and/or additional classification and predictive modeling techniques to correspond to products having multiple specifications (e.g., products regarding denosumab).

In accordance with the above, and with the disclosure herein, the present disclosure includes improvements in computer functionality or in improvements to other technologies at least because the claims recite, e.g., configurable handheld biological analyzer for identification of biological products based on Raman spectroscopy, which are improvements to existing handheld biological analyzers. That is, the present disclosure describes improvements in the functioning of the computer itself or “any other technology or technical field” because the configurable handheld biological analyzers are computing devices, as described herein, and provide, via their biological ensemble classification model configurations, reduced analyzer-to-analyzer variability when compared with existing handheld biological analyzers. This improves over the prior art at least because the configurable handheld biological analyzers described herein provide increased accuracy with respect to measurement, identification, and/or classification of materials and/or products (e.g., therapeutic products), which is important feature in the manufacture and development of pharmaceutical and biological products.

In addition, configurable handheld biological analyzers, as described herein, are further improved by use of the biological ensemble classification model configuration, which is transferable, optionally updatable (with new data), and loadable into a memory of compatible configurable handheld biological analyzer(s), which allows for standardization, and thereby reduced variability, among a set or group (i.e., a “network”) of analyzers. This reduces the maintenance and/or time of deployment for the configurable handheld biological analyzers for the analyzer network.

In addition, the configurable handheld biological analyzer is further improved by use of the biological ensemble classification model configuration, which includes a biological ensemble classification model. The biological ensemble classification model improves the accuracy of identification and/or classification of biological products by eliminating or reducing Type I error (e.g., false positives) and/or Type II error (e.g., false negatives), as described herein.

In addition, the present disclosure includes applying the certain of the claim elements with, or by use of, a particular machine, e.g., a configurable handheld biological analyzer for identification of biological products based on Raman spectroscopy using ensemble AI, including identification of biological products during development or manufacture of such products.

Moreover, the present disclosure includes effecting a transformation or reduction of a particular article to a different state or thing, e.g., transforming or reducing a Raman spectra dataset to different state used for identification of biological products based on Raman spectroscopy.

The present disclosure includes specific features other than what is well-understood, routine, conventional activity in the field, or adding unconventional steps that confine the claim to a particular useful application, e.g., including providing a biological ensemble classification model configuration used for reducing variability among a set or group (i.e., “network”) of configurable handheld biological analyzers that may each by used for identification of biological products based on Raman spectroscopy. Methods and systems described herein can detect and differentiate between product types having similar Raman spectra datasets or otherwise similar Raman related features, but which cannot be differentiated by conventional systems and methods.

Advantages will become more apparent to those of ordinary skill in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### BRIEF DESCRIPTION OF THE DRAWINGS

The Figures described below depict various aspects of the system and methods disclosed therein. It should be understood that each Figure depicts an embodiment of a particular aspect of the disclosed system and methods, and that each of the Figures is intended to accord with a possible embodiment thereof. Further, wherever possible, the following description refers to the reference numerals included in the following Figures, in which features depicted in multiple Figures are designated with consistent reference numerals.

There are shown in the drawings arrangements which are presently discussed, it being understood, however, that the present embodiments are not limited to the precise arrangements and instrumentalities shown, wherein:

FIG. 1 illustrates an example configurable handheld biological analyzer for identification of biological products based on Raman spectroscopy using ensemble artificial intelligence (AI), in accordance with various embodiments disclosed herein.

FIG. 2A illustrates an example biological classification ensemble model for identification of biological products based on Raman spectroscopy using ensemble AI, in accordance with various embodiments disclosed herein.

FIG. 2B illustrates a further example flowchart of a biological analytics method for identification of biological products based on Raman spectroscopy using ensemble AI, in accordance with various embodiments disclosed herein.

FIG. 3A illustrates an example visualization of Raman-based spectra datasets as scanned by various handheld biological analyzers, in accordance with various embodiments disclosed herein.

FIG. 3B illustrates an example visualization of modified Raman-based spectra datasets as modified from the Raman-based spectra datasets of FIG. 3A, in accordance with various embodiments disclosed herein.

FIG. 3C illustrates an example visualization of normalized Raman-based spectra datasets as a normalized version of the modified Raman-based spectra datasets of FIG. 3B, in accordance with various embodiments disclosed herein.

FIG. 4 illustrates an example visualization of Raman-based spectra datasets for mAb 1 (a canonical IgG2 monoclonal antibody) drug product (DP) and mAb 2 (a canonical IgG1 monoclonal antibody) DP as scanned by a handheld biological analyzer, in accordance with various embodiments disclosed herein.

FIG. 5A illustrates an example visualization of Q-residual error of a non-ensemble unsupervised biological classification model when the Raman-based spectra datasets, including those of FIG. 4, are provided as input.

FIG. 5B illustrates an example visualization of predictive output of a supervised biological classification ensemble model, in accordance with various embodiments herein, when the Raman-based spectra datasets, including those of FIG. 4, are provided as input.

FIGS. 6A to 6C illustrate example computer program listings that include pseudo code of a biological ensemble classification model configuration, including configuration for an unsupervised portion of a biological classification ensemble model, in accordance with various embodiments disclosed herein.

FIGS. 7A to 7C illustrate example computer program listings that include pseudo code of a biological ensemble classification model configuration, including configuration for a supervised portion of a biological classification ensemble model, in accordance with various embodiments disclosed herein.

The Figures depict preferred embodiments for purposes of illustration only. Alternative embodiments of the systems and methods illustrated herein may be employed without departing from the principles of the invention described herein.

### DETAILED DESCRIPTION

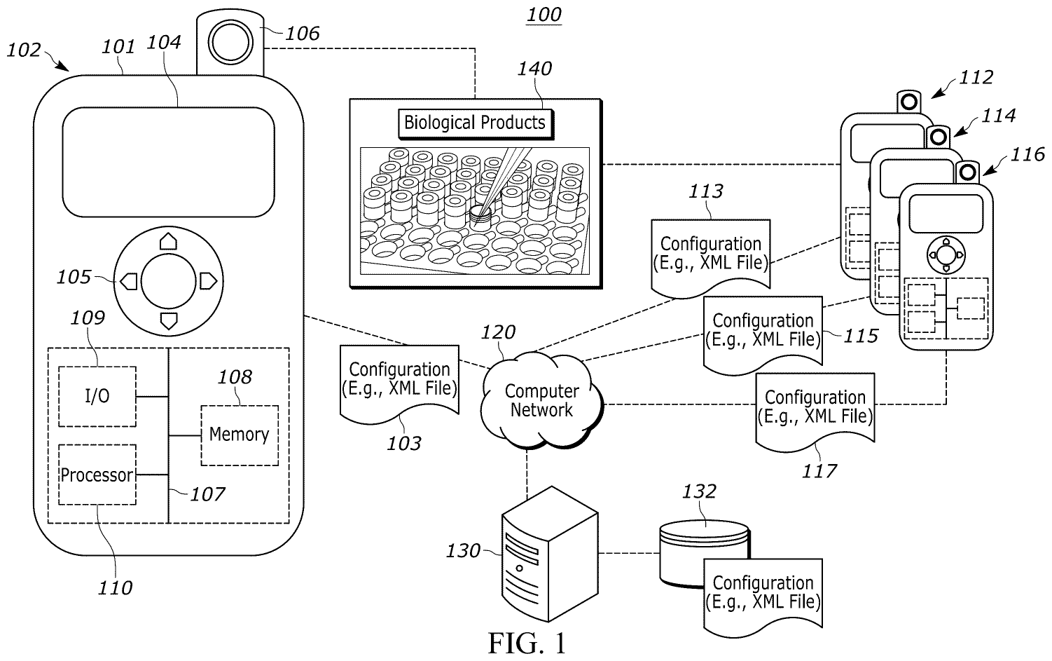

FIG. 1 illustrates an example configurable handheld biological analyzer 102 for identification of biological products 140 based on Raman spectroscopy using ensemble artificial intelligence (AI), in accordance with various embodiments disclosed herein. In the embodiment of FIG. 1, configurable handheld biological analyzer 102 includes first housing 101 molded or otherwise adapted for handheld manipulation. In addition, configurable handheld biological analyzer 102 includes first scanner 106 carried by (e.g., such as coupled to or connected to, directly or indirectly) the first housing. Configurable handheld biological analyzer 102 also includes first processor 110 communicatively coupled to first scanner 106. Configurable handheld biological analyzer 102 may further include first computer memory 108 communicatively coupled to first processor 110. In addition, configurable handheld biological analyzer 102 may include input/output (I/O) component 109 for receiving input from navigation wheel 105. For example, a user may manipulate navigation wheel 105 to select or scroll data or information of a particular sample of a biological product, e.g., as scanned from scanning biological products 140. Input/output (I/O) component 109 may also control display of measurement, identification, classification, or other information as described herein on display screen 104. Each of display screen 104, navigation wheel 105, first scanner 106, first computer memory 108, I/O component 109, and/or first processor 110 are communicatively coupled via electronic bus 107 that is configured to send and/or receive electronic signals (e.g., control signals) or information among the various components, including 104 to 110. In some embodiments, configurable handheld biological analyzer 102 may be a Raman-based handheld analyzer, such as a TruScan™ RM Handheld Raman Analyzer as provided by Thermo Fisher Scientific Inc.

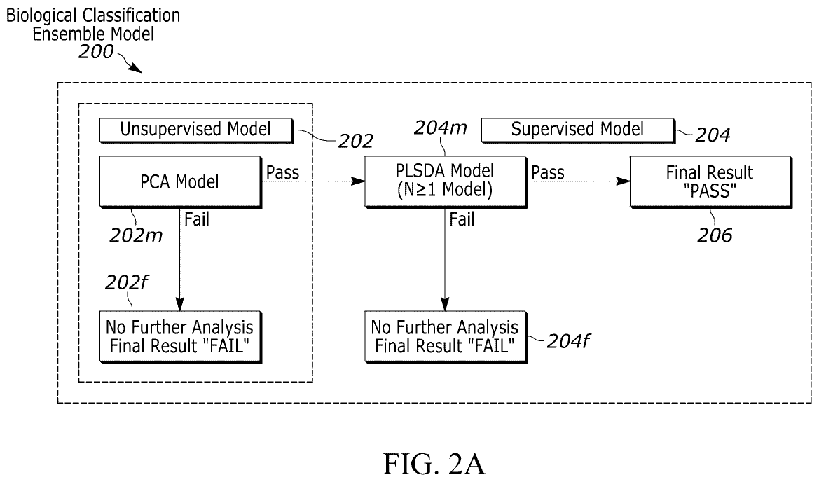

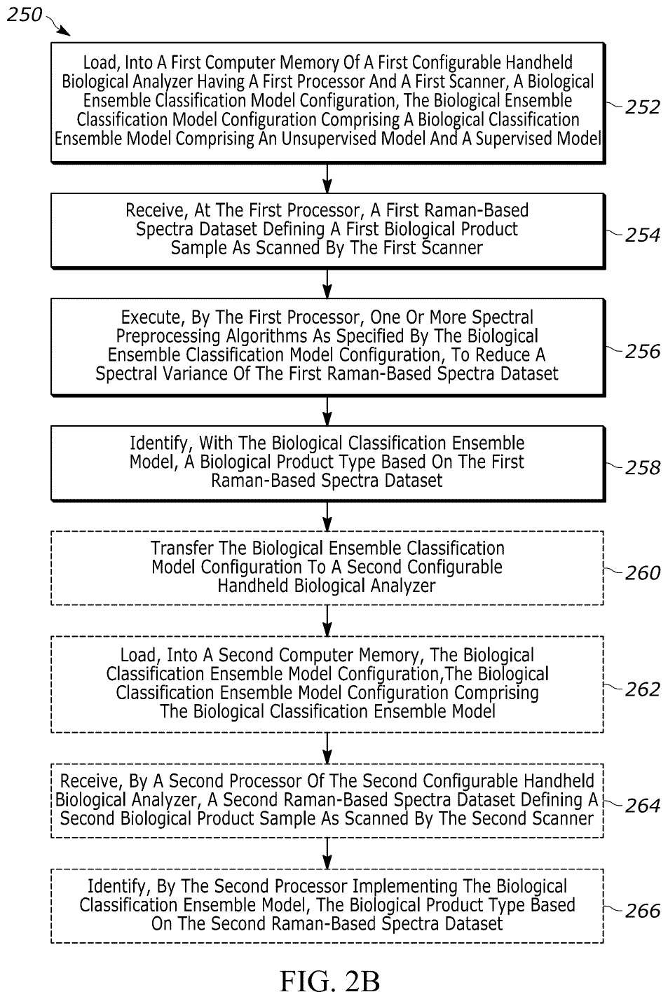

In various embodiments, first computer memory 108 is configured to load a biological ensemble classification model configuration, e.g., biological ensemble classification model configuration 103. Biological classification ensemble model configuration 103 may be used to implement the biological analytics method of FIGS. 2A and/or 2B for identification of biological products based on Raman spectroscopy, as described further herein.

In additional embodiments, computer memory is configured to load a new biological classification ensemble model. The new biological classification model may comprise an updated unsupervised model and/or an updated supervised model trained on a new and/or updated set of Raman spectra data.

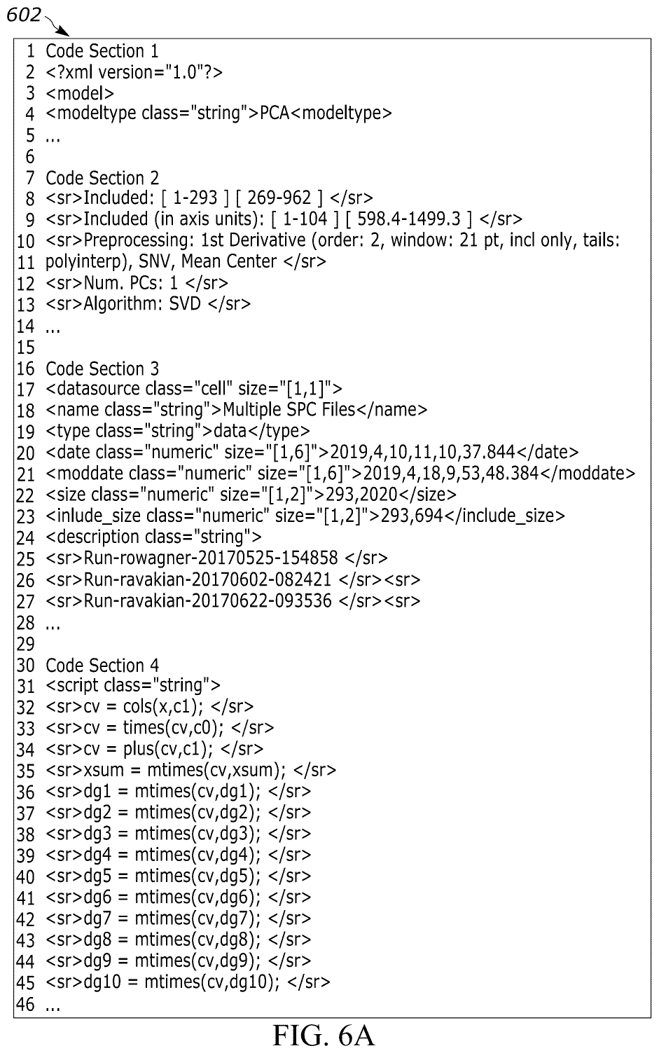

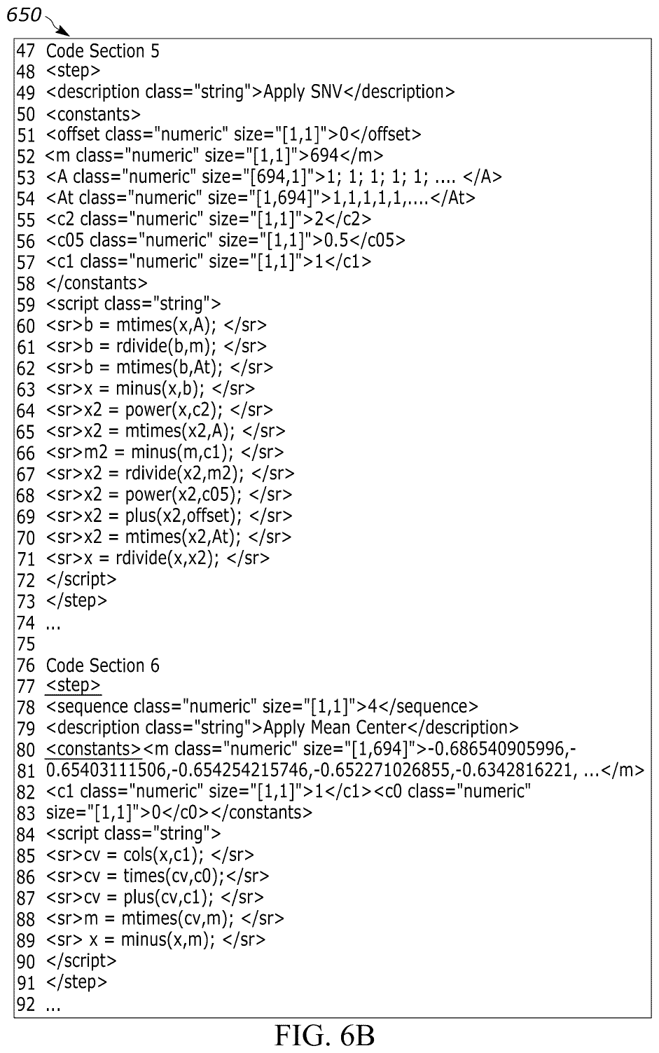

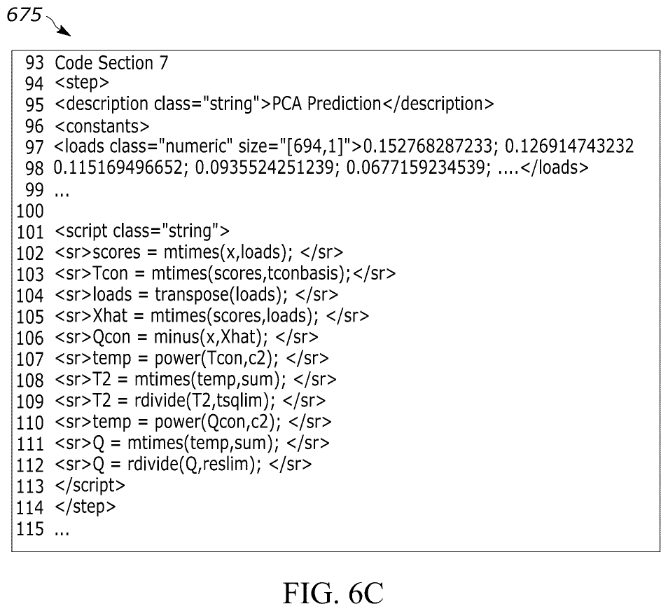

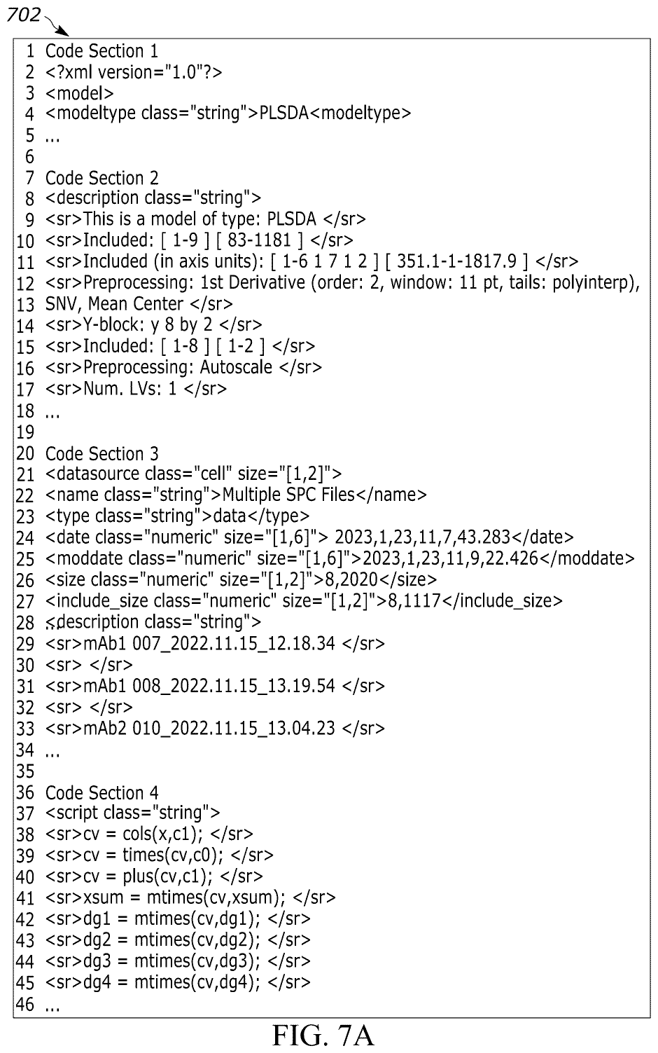

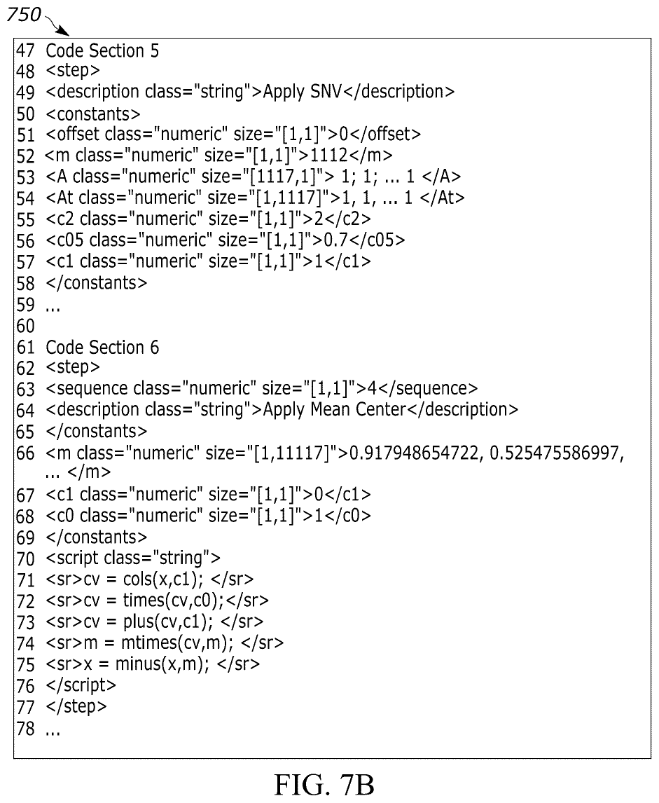

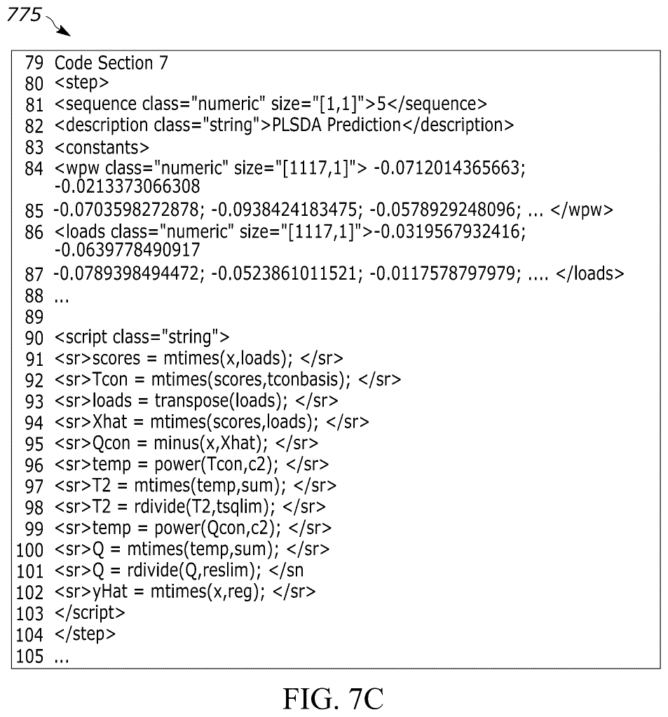

In the embodiment of FIG. 1, biological ensemble classification model configuration 103 is implemented as an extensible markup language (XML) file in an XML format. As described in various embodiments herein, FIGS. 6A to 6C illustrate an example computer program listing, comprising several code portions 602, 650, and 675, that includes pseudo code of a biological ensemble classification model configuration (e.g., biological ensemble classification model configuration 103) in XML format. FIGS. 6A to 6C include example pseudo code for configuration for an unsupervised portion of a biological classification ensemble model. Similarly, FIGS. 7A to 7C illustrate example pseudo code of a biological ensemble classification model configuration, which includes configuration for a supervised portion of a biological classification ensemble model across several code portions comprising code portions 702, 750, and 775. In the embodiment computer program listing of FIGS. 6A to 6C, for example, at Code Section 1, biological ensemble classification model configuration 103 is formatted in XML, where a biological ensemble classification model (“<model>”), or portion thereof, such as unsupervised model 202m of FIG. 2A, is defined within biological ensemble classification model configuration 103. Likewise, in the embodiment computer program listing of FIGS. 7A to 7C, for example, at Code Section 1, biological ensemble classification model configuration 103 is formatted in XML, where a biological ensemble classification model (“<model>”), or portion thereof, such as supervised model 204m of FIG. 2A, is defined within biological ensemble classification model configuration 103. Biological ensemble classification model configuration 103 is transferrable, installable, and/or otherwise implementable or executable on similarly configured configurable handheld biological analyzers (e.g., configurable handheld biological analyzers 112, 114, and/or 116). It is to be understood that the pseudo code of FIGS. 6A-6C and 7A-7C maybe be combinable into a single file or, in the alternative, may be separate files. Such file(s) maybe stored, linked, or/otherwise stored or referenced for access by one or more processors (as described herein) via biological ensemble classification model configuration 103, which is transferrable, installable, and/or otherwise implementable or executable on similarly configured configurable handheld biological analyzers (e.g., configurable handheld biological analyzers 112, 114, and/or 116).

Each of configurable handheld biological analyzers 112, 114, and 116 comprise the same components as configurable handheld biological analyzer 102 such that the disclosure for configurable handheld biological analyzer 102 applies equally to each of configurable handheld biological analyzers 112, 114, and 116. Each of configurable handheld biological analyzers 102, 112, 114, and 116 may be part of a same analyzer group or set (i.e., comprising an analyzer “network” or group). In some embodiments, each of configurable handheld biological analyzers 102, 112, 114, and/or 116 may have a same, similar, and/or different mix of characteristics or features, such as a same, similar, and/or different mix of software version(s) or type(s), manufacture(s), age(s), operating environment(s) (e.g., temperature), component(s), or other such similarities or differences of Raman-based analyzers.

Regardless of the same, similar, and/or different mix of characteristics or features among configurable handheld biological analyzers 102, 112, 114, and 116, biological ensemble classification model configuration 103, and its related biological ensemble classification model, allows for the network of configurable handheld biological analyzers (e.g., configurable handheld biological analyzers 102, 112, 114, and 116) to yield consistent results when measuring or identifying pharmaceutical or biological product (e.g., therapeutic products/drugs). That is, despite the similarities or differences of a given analyzer network of configurable handheld biological analyzers, such configurable handheld biological analyzers may accurately identify or measure a given pharmaceutical or biological product when such configurable handheld biological analyzers are configured with a biological ensemble classification model configuration as describe herein.

In various embodiments, multiple analyzers may be used to generate or construct a biological ensemble classification model configuration 103 and its related biological ensemble classification model. For example, in some embodiments, any one or more of configurable handheld biological analyzers 102, 112, 114, and 116, and/or other analyzers (not shown) may be used to generate or construct a biological ensemble classification model.

Generation of a biological ensemble classification model configuration 103, and its related biological ensemble classification model, generally requires a group or network of analyzers scanning samples (e.g., of biological products 140) to produce Raman-based spectra datasets of those samples. For example, scanning biological products 140, e.g., by any of configurable handheld biological analyzers 102, 112, 114, and 116, can yield detailed information regarding biological products 140. For example, the detailed information can include Raman-based spectra dataset(s) defining a biological product sample(s) (e.g., of biological products 140). Examples of biological products 140 may include any of mAb 3 DP, mAb 2 drug substance (DS), mAb 1 DP, and/or as otherwise as described herein. However, it is to be understood that additional biological products are contemplated herein, and biological products 140 are not limited to any specific biological product or grouping thereof.

In some embodiments, configurable handheld biological analyzer 102 may define instrument or analyzer-based spectral acquisition parameters (e.g., integration time, laser power, etc.) to be used for scanning samples, e.g., of biological products 140. For example, a user, via navigation wheel 105 may select the spectral acquisition parameters to use of scanning a sample. In some embodiments, configurable handheld biological analyzer 102 may generate an output file (e.g., an output file of the “.acq” file type) that specifies the spectral acquisition parameters.

In some embodiments, a configurable handheld biological analyzer (e.g., configurable handheld biological analyzer 102) may load a file (e.g., an “.acq” file) to configure the configurable handheld biological analyzer with the spectral acquisition parameters to use for scanning a target product. As described herein, Raman-based spectra dataset(s) may be scanned, by one or more configurable handheld biological analyzer(s) (e.g., configurable handheld biological analyzer 102), in order to generate a biological ensemble classification model configuration (e.g., biological ensemble classification model configuration 103). In some embodiments, sample(s) (e.g., multiple lots) of a biological product (e.g., of biological products 140) may be selected as a representative target product for scanning. Generally, a “target product,” as described herein, represents a biological product used to train or otherwise configure a biological ensemble classification model configuration and its related model. Generally, a target product is selected based on its biological specifications. Once setup with the spectral acquisition parameters to use for scanning a target product, a configurable handheld biological analyzer (e.g., configurable handheld biological analyzer 102) may scan (e.g., with first scanner 106) samples of the target product, in some cases multiple times (e.g., fourteen (14) times)), where each scan generates detailed information, including Raman-based spectra dataset(s) of the target product.

In a similar embodiment, multiple configurable handheld biological analyzers (configurable handheld biological analyzers 102, 112, 114, and/or 116)) may load the output file (e.g., “.acq” file) to setup each configurable handheld biological analyzer with the spectral acquisition parameters to use for scanning biological product samples. Once setup, each configurable handheld biological analyzer (e.g., any of configurable handheld biological analyzers 102, 112, 114, and/or 116) is configured to scan (e.g., with first scanner 106) the samples, in some cases multiple times (e.g., fourteen (14) times)), where each scan generates detailed information, including Raman-based spectra dataset(s), of the target product. By scanning a given target product with different/multiple scanners, the Raman-based spectra dataset(s) captured by those scanners become robust in that the Raman-based spectra dataset(s) capture any differences (e.g., caused by software, manufacture, age, operating environment (e.g., temperature), etc.) among the scanners. In this way, the Raman-based spectra dataset(s) provide an ideal training dataset for reducing variability among the multiple scanners as described herein. Each of the Raman-based spectra dataset(s), e.g., as scanned by the multiple scanners (e.g., any of configurable handheld biological analyzers 102, 112, 114, and/or 116), may be output and/or saved as a Raman spectrum file, for example, having a “.spc” file type.

It is to be understood that Raman-based spectra dataset(s) may also be captured for a challenge product in the same or similar manner as for a target product. As used herein, a “challenge product” describes a biological product (e.g., selected from biological products 140) that a configurable handheld biological analyzer (e.g., configurable handheld biological analyzer 102) is configured to identify, classify, or measure, when loaded or otherwise configured with a biological ensemble classification model configuration (e.g., biological ensemble classification model configuration 103) and its related biological ensemble classification model, as described herein.

Raman-based spectra dataset(s) for a challenge product may be captured in the same/or similar manner as for a target product, where a challenge product may be selected based on its biological specifications and where the a configurable handheld biological analyzer (e.g., configurable handheld biological analyzer 102) may load an output file (e.g., “.acq” file) to configure the configurable handheld biological analyzer with the spectral acquisition parameters to use for scanning the challenge product. Once setup, the configurable handheld biological analyzer (e.g., configurable handheld biological analyzer 102) is configured to scan (e.g., with first scanner 106) the samples of the challenge product, in some cases multiple times (e.g., three (3) times)), where each scan generates detailed information, including Raman-based spectra dataset(s) of the challenged product. The Raman-based spectra dataset(s), e.g., as scanned by the configurable handheld biological analyzer 102, may be output and/or saved as a Raman spectrum file, for example, having a “.spc” file type.

In some embodiments, generation of a biological ensemble classification model configuration (e.g., biological ensemble classification model configuration 103) may be performed by a remote processor, such as a processor of computer 130 illustrated by FIG. 1. For example, Raman-based spectra dataset(s), as generated for a biological product (e.g., selected from biological products 140) as described herein, may be imported into and/or analyzed by modeling software, executing on computer 130, configured to analyze Raman-based spectra dataset(s). One example of such modeling software includes SOLO (stand-alone chemo-metrics software) as provided by Eigenvector Research, Inc. However, it is to be understood that other modeling software, including custom or proprietary software, implemented to perform the features described herein may also be used. The modeling software may build or generate a biological ensemble classification model based on the Raman-based spectra dataset(s). For example, in some embodiments, Raman-based spectra dataset(s) as scanned or captured for target or challenge product(s), as described herein, may be used to build or generate a biological ensemble classification model. Still further, Raman-based spectra dataset(s) (e.g., for a target product or a challenge product) may also be used for cross validation of the biological ensemble classification model. For example, Raman-based spectra dataset(s) may be used to evaluate Type I error (e.g., false positives) and Type II error (e.g., false negatives) of a biological ensemble classification model against cross validation data set of Raman-based spectra dataset(s).

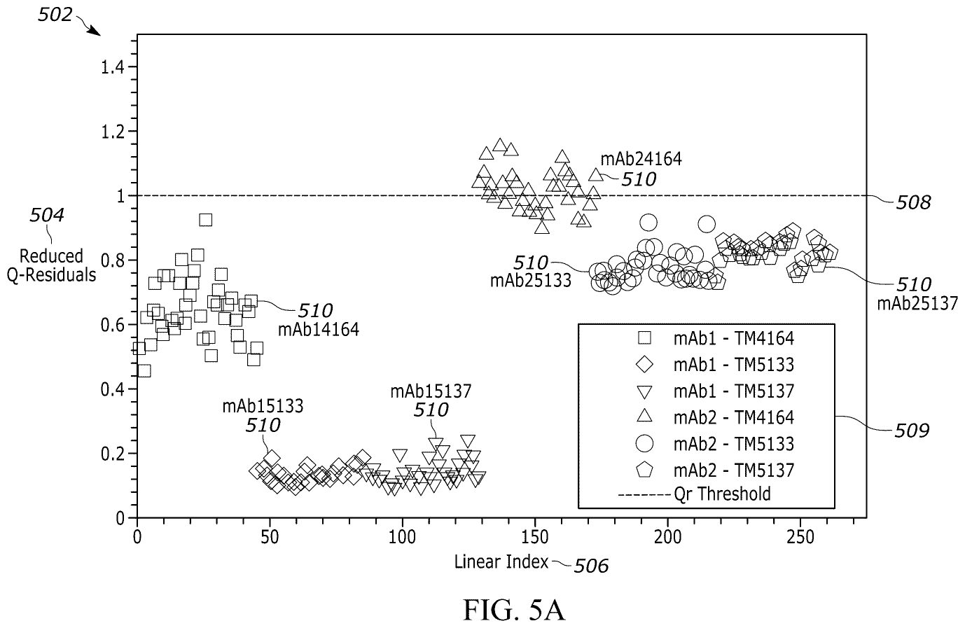

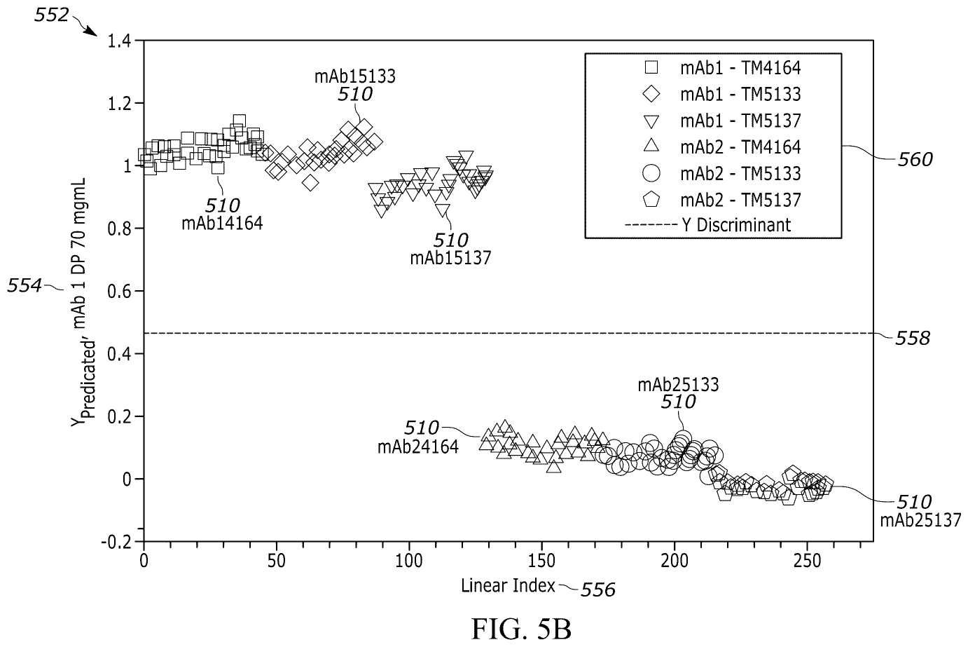

In various embodiments, biological ensemble classification model, and/or its related biological ensemble classification model configuration (e.g., biological ensemble classification model configuration 103), may be generated to include algorithms (e.g., scripts) and parameters to be used by a configurable handheld biological analyzer (e.g., configurable handheld biological analyzer 102) to identify, classify, and/or measure biological products as described herein. Examples of the algorithms (e.g., scripts) and/or parameters are described with respect to FIGS. 2A, 2B, 6A-6C, and 7A-7C herein. For example, a biological ensemble classification model configuration (e.g., biological ensemble classification model configuration 103) may include parameters defining details of the biological ensemble classification model. For example, such parameters may include the number of classification components of the biological ensemble classification model, loadings, etc. As term is used herein a “classification component” may comprise a principal component determined of a principal component analysis (PCA). In other embodiments, more generally, a classification component can be a coefficient or variable of multivariate model (such as a regression model or machine learning model). Based on the classification component, the biological ensemble classification model is configured to identify the biological product type of a given biological product sample (e.g., selected from biological products 140). For example, in one embodiment, the number of classification components may be determined, e.g., by modeling software, through singular value decomposition (SVD) analysis where the classification components comprise one or more principal components of a PCA. A PCA implementation represents use of multivariate analysis, e.g., as implemented by configurable handheld biological analyzer 102 configured with biological ensemble classification model configuration 103, for distinguishing biological products (e.g., biological products 140), such as therapeutic products/drugs having similar formulations (e.g., as describe herein for FIGS. 5A and 5B). For example, biological or pharmaceutical products are typically associated with high-dimensional data. High-dimensional data can include multiple features, such as expression of many genes, measured a given sample (e.g., a sample of scanning biological products 140). PCA provides a technique, as used by configurable handheld biological analyzer 102, to simplify complexity in high-dimensional data (e.g., Raman spectra dataset(s)) while retaining trends and patterns that are useful for predictive and/or identification purposes (e.g., identifying biological products as describe herein). For example, application of PCA includes transforming (e.g., by first processor 110) a dataset (e.g., a Raman-based spectra dataset) into fewer dimensions. A transformed dataset with fewer dimensions provides a summary or simplification of the original dataset. The transformed dataset, in turn, reduces computational expense when manipulated by a configurable handheld biological analyzer (e.g., configurable handheld biological analyzer 102) described herein. Further, error rate(s), as described herein, may also be reduced by implementing PCA thereby eliminating the need to apply test correction(s) to data of a higher-dimension when testing each feature for association with a particular outcome.

In addition, PCA, as implemented by configurable handheld biological analyzer 102, reduces data complexity by geometrically projecting them onto lower dimensions called principal components (PCs), and by targeting the best summary of the data, and therefore PCs, by using a limited number of PCs. A first PC is chosen to minimize the total distance between the data and their projection onto the PC. Any second (subsequent) PCs are selected similarly, with the additional requirement that they be uncorrelated with all previous PCs.

PCA is an unsupervised learning method and is similar to clustering—it finds trends or patterns without reference to prior knowledge about whether the samples come from different sources, such as different configurable handheld biological analyzers (e.g., configurable handheld biological analyzers 102, 112, 114, and/or 116). For example, in some embodiments, a classification component, of a biological ensemble classification model, may be a first principal component of a PCA model. In such embodiments, the first principal component may be determined, by first processor 110, based on a singular value decomposition (SVD) analysis. Use of a first principal component, by configurable handheld biological analyzer 102, limits or reduces the amount of analyzer variability accounted for by its biological ensemble classification model. In some embodiments, the first principal component (PC) may be the only principal component. In other embodiments, a biological ensemble classification model may comprise a second classification component, where a biological ensemble classification model is configured to identify biological product type(s) of a given biological product sample (e.g., biological products 140) based on multiple classification components (e.g., the first classification component and the second classification component).

The modeling software may be configured to set statistical confidence levels to determine the classification components (e.g., principal components) for inclusion in, or otherwise use by, the biological ensemble classification model. For example, in the embodiment of computer program listing of FIGS. 6A to 6C, at Code Section 1, the biological ensemble classification model configuration indicates that a biological ensemble classification model (e.g., the defined “<model>”) comprises a PCA type of biological ensemble classification model. This indicates that the classification components of the biological ensemble classification model will comprise principal components. For example, in the embodiment of FIGS. 6A to 6C, Code Section 2 indicates the number of principal components is to be one (single) principal component (“Num. PCs: 1”) that is to be determined via an SVD analysis (“Algorithm: SVD”) to be executed, for example, on first processor 110 of configurable handheld biological analyzer 102. Similarly, the modeling software may be configured to set statistical values to determine an equation (e.g., best-fit or least squares linear equation) for inclusion in, or otherwise use by, the biological ensemble classification model. For example, in the embodiment computer program listing of FIGS. 7A to 7C, at Code Section 1, the biological ensemble classification model configuration indicates that a biological ensemble classification model (e.g., the defined “<model>”) comprises a PLSDA type of biological ensemble classification model. This indicates that the classification values of the biological ensemble classification model will comprise PLSDA values. For example, in the embodiment of FIGS. 7A to 7C, Code Section 2 indicates the model comprises a PLSDA model having certain “axis units,” Y-block values, and linear values (“Num. LVs: 1”) that is to be determined via an PLSDA model to be executed, for example, on first processor 110 of configurable handheld biological analyzer 102.

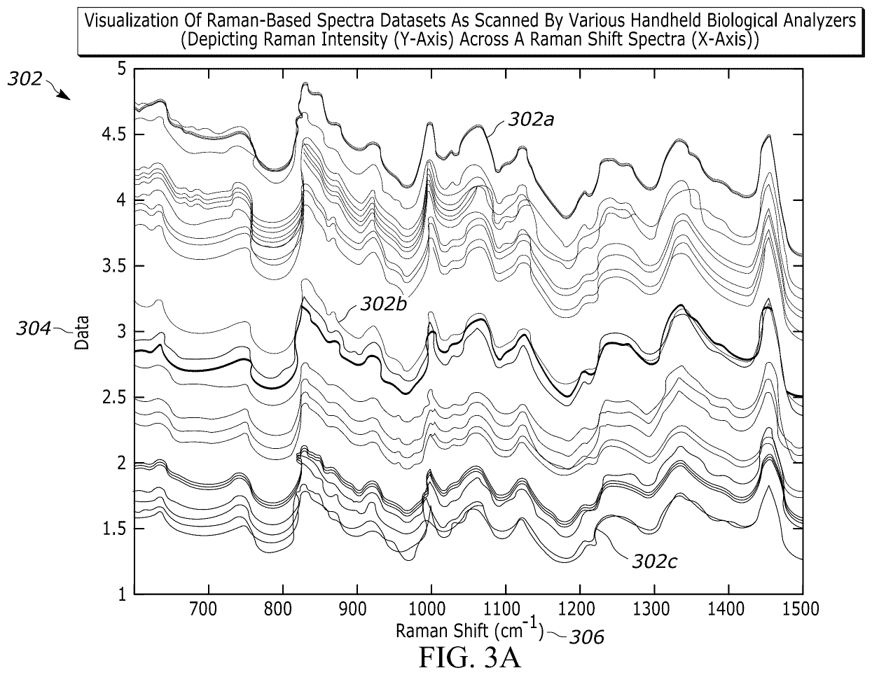

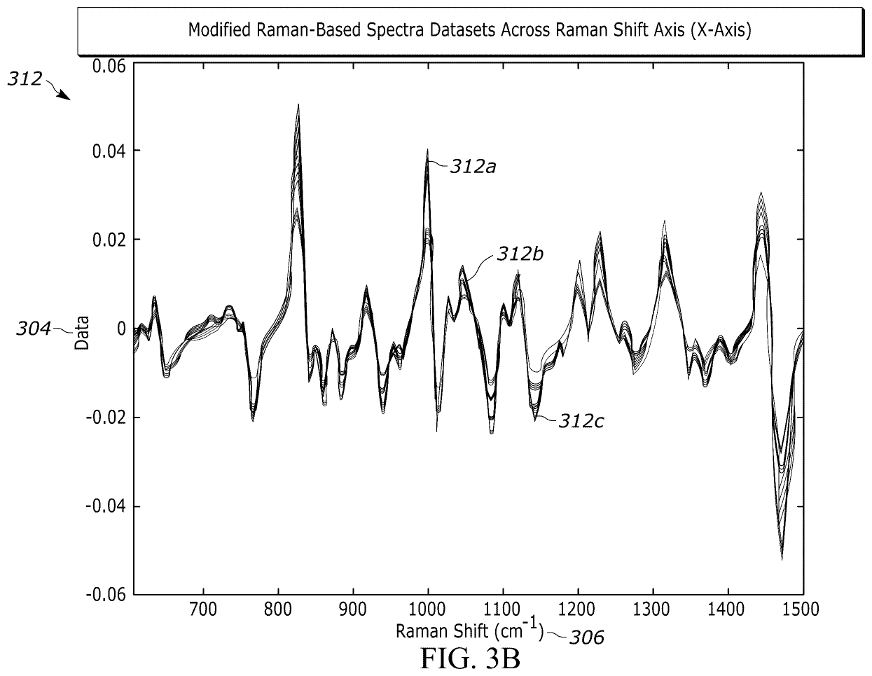

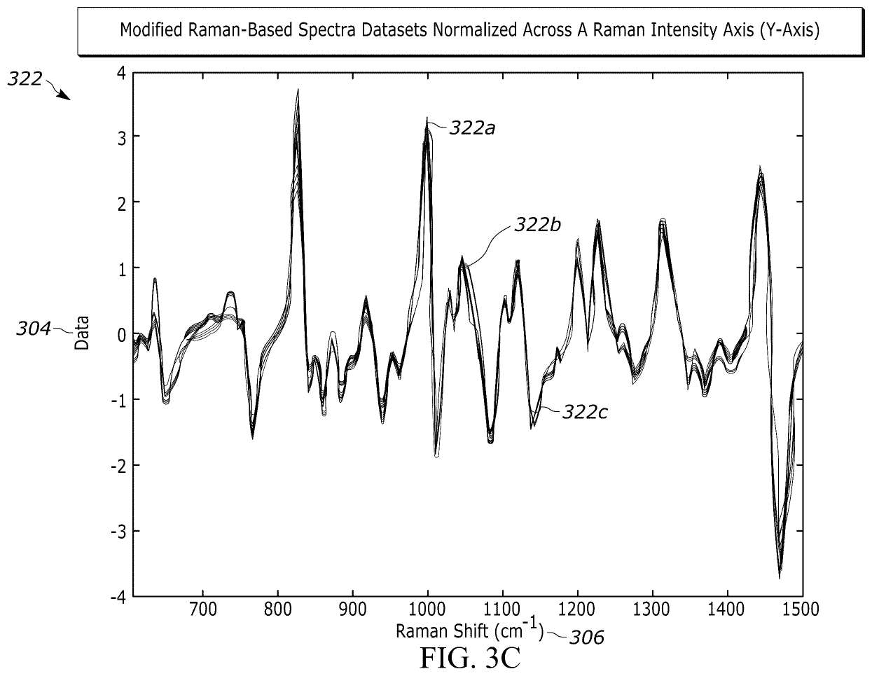

As a further example, a biological ensemble classification model configuration (e.g., biological ensemble classification model configuration 103) may include computer code or scripts for defining or implementing spectral preprocessing algorithm(s), for example, as described with respect to FIGS. 3A to 3C. For example, first processor (e.g., first processor 110) may be configured to execute the one or more spectral preprocessing algorithms to reduce a spectral variance of a first Raman-based spectra dataset when the first Raman-based spectra dataset is received by the first processor. More generally, the computer code or scripts for defining or implementing spectral preprocessing algorithm(s) may be executed on a processor (e.g., first processor 110), where the processor receives Raman-based spectra dataset(s) of biological products (e.g., biological products 140). The configurable handheld biological analyzer then executes the computer code or scripts defining or implementing spectral preprocessing algorithm(s) to prepare/preprocess the data for input into classification component(s) of the biological ensemble classification model in order to identify, measure, or classify a biological product (e.g., a challenge product) as described herein. For example, in the embodiment computer program listing of FIGS. 6A to 6C, at Code Section 2, the biological ensemble classification model configuration includes an execution sequence of an example spectral preprocessing algorithm (e.g., “Preprocessing: 1st Derivative (order: 2, window: 21 pt, incl only, tails: polyinterp), SNV, Mean Center”), which includes determining a first derivative, applying a standard normal variate (SNV) algorithm, and further applying a meaning centering function to a Raman-based spectra dataset scanned for a particular product (e.g., target product or challenge product). Code Section 2 of FIGS. 7A-7C show a similar embodiment with respect to the PLSDA model. Further, an example embodiment of this execution sequence is described and visualized herein with respect to FIGS. 3A to 3C and Code Sections 4 to 6 of FIGS. 6A to 6C and FIGS. 7A to 7C.

As a further example, a biological ensemble classification model configuration (e.g., biological ensemble classification model configuration 103) may include the Raman-based spectra dataset(s) used to generate the biological ensemble classification model. For example, in the embodiment computer program listing of FIGS. 6A to 6C, at Code Section 3, the biological ensemble classification model configuration (e.g., biological ensemble classification model configuration 103) includes example Raman-based spectra dataset(s) used to generate the biological ensemble classification model, or portion thereof, in accordance with the pseudo code of FIGS. 6A to 6C. Similarly, in the embodiment computer program listing of FIGS. 7A to 7C, at Code Section 3, the biological ensemble classification model configuration (e.g., biological ensemble classification model configuration 103) includes example Raman-based spectra dataset(s) used to generate the biological ensemble classification model, or portion thereof, in accordance with the pseudo code of FIGS. 7A to 7C.

In some embodiments, the biological ensemble classification model configuration (e.g., biological ensemble classification model configuration 103) may also define threshold values, for example as statistical acceptance criteria, to determine whether a biological product has been successfully identified or measured by a configurable handheld biological analyzer 102. For example, such threshold values may define pass/fail thresholds for Q-residuals values (e.g., as described herein for FIGS. 2A, 5A, and 5B) to determine whether a biological product has been successfully identified or measured by configurable handheld biological analyzer 102. In other embodiments, the threshold values may be configured independently from the biological ensemble classification model configuration (e.g., biological ensemble classification model configuration 103), for example, by the user configuring and/or defining the threshold values manually via the navigation wheel 105 and display screen 104 described herein.

Once generated, a biological ensemble classification model and its related biological ensemble classification model configuration (e.g., biological ensemble classification model configuration 103) may be exported to a file (e.g., an XML file, as described herein) for transmission (e.g., via computer network 120 or otherwise described herein) to, and/or for loading into the memory of, configurable handheld biological analyzers (e.g., any one or more of configurable handheld biological analyzers 102, 112, 114, and/or 116). In some embodiments, output file(s) (e.g., an “.acq” file as describe herein), may also be transmitted to (e.g., via computer network 120 or otherwise described herein), and/or loaded into the memory of, configurable handheld biological analyzers (e.g., any one or more of configurable handheld biological analyzers 102, 112, 114, and/or 116).

A biological ensemble classification model may be generated by a remote processor that is remote to a given configurable handheld biological analyzer. For example, in the embodiment of FIG. 1, computer 130 includes a remote process that is remote to configurable handheld biological analyzer 102. Computer 130 may generate (e.g., as described herein) and store one or more biological ensemble classification model configuration(s) and/or biological ensemble classification models in database 132. In various embodiments, computer 130 may transfer, over computer network 120, biological ensemble classification model configuration(s) (e.g., any of biological ensemble classification model configurations 103, 113, 115, and/or 117) to a configurable handheld biological analyzers (e.g., to configurable handheld biological analyzers 102, 112, 114, and/or 116, respectively). In some embodiments, each of biological ensemble classification model configurations 103, 113, 115, and/or 117 may be copies of a same file (e.g., same XML file). Computer network 120 may comprise a wired and/or wireless (e.g., 802.11 standard network) implementing a computer packet protocol, such as, for example transmission control protocol (TCP)/internet protocol (IP). In other embodiments, a biological ensemble classification model configuration (e.g., biological ensemble classification model configuration 103) may be transferred via a universal serial bus (USB) cable (not shown), memory drive (e.g., a flash or thumb drive) (not shown), a disk (not shown), or other transfer or memory device cable of transferring a data file, such as the XML file disclosed herein. In still further embodiments, biological ensemble classification model configuration 103 may be transferred via a wireless standard or protocol, such as Bluetooth, WiFi, or a cellular standard, such as GSM, EDGE, CDMA, and the like.

A biological ensemble classification model configuration (e.g., biological ensemble classification model configuration 103) may be transferred among configurable handheld biological analyzers. Once transferred, a biological ensemble classification model configuration may be loaded into the memory of a configurable handheld biological analyzer to calibrate or configure that configurable handheld biological analyzer to have a reduced variability with respect to other configurable handheld biological analyzers implementing or executing the biological ensemble classification model. For example, in one embodiment, biological ensemble classification model configuration 103 may include a biological ensemble classification model. The biological ensemble classification model of biological ensemble classification model configuration 103 may be configured to execute on first processor 110. For example, first processor 110 may be configured to (1) receive a first Raman-based spectra dataset defining a first biological product sample (e.g., of scanning biological products 140) as scanned by the first scanner, and (2) identify, with the biological ensemble classification model, a biological product type based on the first Raman-based spectra dataset. For example, in some embodiments, the biological product type may be of a therapeutic product having a therapeutic product type. Still further, in some embodiments, the biological product type may be identified by the biological classification ensemble model during manufacture of a biological product having the biological product type. Manufacture of such biological product(s) may comprise processing and storage of a drug product as well as bioreactor production.

The biological ensemble classification model of biological ensemble classification model configuration 103 may be electronically transferred, e.g., via biological ensemble classification model configuration 113 over computer network 120 to configurable handheld biological analyzer 112. Just as for configurable handheld biological analyzer 102, configurable handheld biological analyzer 112 may comprise a second housing adapted for handheld manipulation, a second scanner coupled to the second housing, a second processor communicatively coupled to the second scanner, and a second computer memory communicatively coupled to the second processor. The second computer memory of configurable handheld biological analyzers 112 is configured to load the biological ensemble classification model configuration 113. Biological ensemble classification model configuration 113 includes the biological ensemble classification model of biological ensemble classification model configuration 103. When implemented or executed on the second processor of configurable handheld biological analyzer 112, the second processor is configured to (1) receive a second Raman-based spectra dataset defining a second biological product sample (e.g., taken from scanning biological products 140) as scanned by the second scanner of configurable handheld biological analyzer 112, and (2) identify, with the biological ensemble classification model, the biological product type based on the second Raman-based spectra dataset. In such embodiments, the same biological product or product type may be identified, by use of the same biological ensemble classification model, as transferred by the biological ensemble classification model configuration files, where the second biological product sample is a new sample of the biological product type (e.g., the same biological product type as analyzed by the first configurable handheld biological analyzer 102).

In various embodiments, new or additional Raman-based spectra dataset(s) may be scanned by configurable handheld biological analyzers and used to update a biological ensemble classification model. In such embodiments, an updated biological ensemble classification model may be transferred to a configurable handheld biological analyzer (e.g., configurable handheld biological analyzer 102) as described herein.

In some embodiments, the computer memory (e.g., first computer memory 108) of a configurable handheld biological analyzer (e.g., configurable handheld biological analyzer 102) may be configured to load a new biological ensemble classification model where the new biological ensemble classification model may comprise an updated classification component. The new classification component may be, for example, generated or determined for a new biological ensemble classification model configuration 103 as received with a new biological ensemble classification model configuration (e.g., biological ensemble classification model configuration 103).

As described in various embodiments herein, a configurable handheld biological analyzer (e.g., configurable handheld biological analyzer 102) may be configured by loading the biological ensemble classification model configuration, and its related biological ensemble classification model. Once configured, configurable handheld biological analyzer 102 may be used to identify, classify, or measure products of interest (e.g., challenge products and/or samples), as described herein.

FIG. 2A illustrates an example biological classification ensemble model 200 for identification of biological products based on Raman spectroscopy, in accordance with various embodiments disclosed herein. As shown for FIG. 2A, biological classification ensemble model 200 comprises an unsupervised model 202 and a supervised model 204. In the example of FIG. 2A, unsupervised model 202 is configured based on a principal component analysis (PCA) and supervised model 204 is configured on a partial least squares discriminant analysis (PLSDA). A configured model is a model that is trained on data (e.g., Raman-spectra data), and where the trained model may be used for predictive, classification, or otherwise product identification purposes thereafter. In various aspects, the models may be independent, where each may be independently trained or otherwise configured on the same and/or different Raman-based spectra training data, and where each model has its own indicator output (e.g., pass-fail is independently determined and/or output). For example, contemplated herein are multiple models, for example two, three, or more models, each with its own indicator output.

A biological classification ensemble model (e.g., biological classification ensemble model 200) may be configured to identify a biological product type upon determination that a first indicator passes a first pass-fail based threshold value and that a second indicator passes a second pass-fail based threshold value. For example, as shown in the example of FIG. 2A, biological classification ensemble model 200 may comprise chained or otherwise sequential outputs, where one model passes its output to the other in order to identify (e.g., PASS or FAIL) a particular target product (e.g., a drug product). In this way, a biological a configurable handheld biological analyzer 102, having a biological classification ensemble model 200 loaded thereon, may be implemented, executed, or otherwise accessed to identify (e.g., PASS or FAIL) a particular target product (e.g., a drug product).

Unsupervised model 202 may comprise an artificial model trained on and/or implementing principal component analysis (PCA), Euclidean distance or correlation, neighbor-based training or implementation, K-means training or implementation, Quality Threshold (QT) training or implementation, Centroid training or implementation, Ward's, and/or Fuzzy C-Means clustering. In various aspects, unsupervised model 202 may be trained with Raman-based spectra training data to configure the unsupervised model to output a first indicator (e.g., a pass-fail indicator) of one or more biological product types (e.g., e.g., one or more target products).

In the example of FIG. 2A, unsupervised model 202 is a PCA model 202m or otherwise a clustering model configured to classify Raman-based spectra data. For example, unsupervised model 202 may comprise a PCA model configured to implement a dimension reduction clustering algorithm that summarizes important variability in a dataset using principal components. The principal components may be used to identify target products, even though Raman spectra are typically highly multicollinear. In particular. the PCA model can be generated or build using Raman spectra of a target product using different sample lots and instruments, where a few principal components (e.g., 1-2 principal components) may be identified that summarize important variability (generally, instrument variability and lot-to-lot differences) with respect to Raman spectra. The data used to identify may comprise column-based values, e.g., variables in MVA literature (for Raman, 1000 or more intensity measurements) and row-based values, e.g., sample spectra, generally a few or tens of samples. Unsupervised model 202 is configured to differentiate or otherwise identify products that differ slightly in protein concentration, formulation, or other difference (e.g., 100 mg/ml mAb 2 differing from 90 mg/ml mAb 3 (a canonical IgG2 monoclonal antibody that is a different antibody than mAb 2)).

Unsupervised model 202 is provided as input Raman-based spectra data (e.g., as related to a test or challenge product) and outputs a pass or fail indicator. The Raman-based spectra data may be data indicative of a particular biological product (e.g., of biological products 140), and an output of FAIL is provided if unsupervised model 202 fails to detect the particular biological product. Such FAIL output may be produced if the unsupervised model 202 produces a value above or below a threshold for the particular biological product, for example, as described herein for FIGS. 5A and 5B. In the example shown in FIGS. 5A and 5B, a FAIL would result from a Q-residual value greater than 1.

With reference to FIG. 2A, if a FAIL result 202f is produced, then no further analysis need be performed, and biological classification ensemble model 200 may produce a failure or negative output as a result. On the other hand, if a pass result is produced, biological classification ensemble model 200 may initiate execution of supervised model 204.

Supervised model 204 may comprise an artificial model trained on and/or implementing partial least squares discriminant analysis (PLSDA), linear discriminant analysis (LDA), K-nearest neighbor (KNN) analysis, soft independent modeling of or by class analogy (SIMCA), and/or logistic regression discriminant analysis (LREGDA). In various aspects, the supervised model 204 may be trained with Raman-based spectra training data to configure the supervised model to output a second indicator (e.g., a pass-fail indicator) of the one or more biological product types.

In the example of FIG. 2A, supervised model 204 is a PLSDA model 204m, e.g., a linear classification model configured to determine a label value indicative of the particular product type. PLSDA model 204m may execute or implement a dimension reduction classification algorithm that summarizes or otherwise detects important variability in a dataset using latent variables. The PLSDA model 204m may be trained to identify or choose latent variables that maximize covariance i.e., Cov (X,Y), between the data block (X) and class label (Y) matrices. In various aspects, X values may comprise Raman spectra, e.g., a data matrix (M×N) having approximately 30×2048 data values. The Y values may comprise a class matrix (N×1), where, for example:
Yᵢ=1 if the sample is the target product; or
Yᵢ=0 if the sample is not the target product

The data may comprise Raman-based spectra data for a given target product and/or the target product with challenge samples having similar values. This allows the supervised model 204 to discover latent variables that distinguish between or among the target product and the challenge samples of similar values. In this way, supervised model 204 is configured to differentiate or otherwise identify products having nearly identical formulations, protein concentrations, molecule classes, and/or other similar attributes.

In some aspects, multiple PLSDA models may be used (not shown) for biological classification ensemble model 200 of FIG. 2A). For example, biological classification ensemble model 200 may be configured or updated to have two supervised models (multiclass models). In such aspects, biological classification ensemble model 200 may be configured or updated to employ models of smaller scope in order to reduce complex datasets and provide streamlined linear discriminants between the target and challenge Raman spectra datasets.

With further reference to FIG. 2A, as for unsupervised model 202, supervised model 204 is provided as input the Raman-based spectra data (e.g., as related to a test or challenge product) and outputs a pass or fail indicator. The Raman-based spectra data may be data indicative of a particular biological product (e.g., of biological products 140). An output of FAIL is provided if supervised model 204 fails to identify or detect the particular biological product. Such FAIL output may be produced if the supervised model 204 produces a value below a threshold or confidence value for the particular biological product, for example, as described herein for FIGS. 5A and 5B.

With further reference to FIG. 2A, if a FAIL result 204f is produced, then no further analysis need be performed, and biological classification ensemble model 200 may produce a failure or negative output (e.g., “0” or FALSE) as a result. On the other hand, if a pass result is produced, biological classification ensemble model 200 outputs a positive (PASS) or otherwise positive output (e.g., “1” or TRUE).

Even though specific models are exemplified for FIG. 2A, it should be understood that the biological classification ensemble model 200 is not limited to a PCA and PLSDA model. For example, in one embodiment a class of powerful, albeit computationally expensive nonlinear methods (all discriminant analyses with the suffix DA) may be used comprising a support vector machine (SVM), artificial neural network (ANN), extreme gradient boosting (XGB). Additionally, or alternatively, an additional embodiment, KNN or LDA is contemplated in combination with PCA for complementary and/or effective results may be used.

FIG. 2B illustrates a further example flowchart of a biological analytics method 250 for identification of biological products based on Raman spectroscopy using ensemble artificial intelligence (AI), in accordance with various embodiments disclosed herein. At block 252, biological analytics method 250 comprises loading, into a first computer memory of a first configurable handheld biological analyzer (e.g., configurable handheld biological analyzer 102) having a first processor (e.g., first processor 110) and a first scanner (e.g., first scanner 106), a biological ensemble classification model configuration (e.g., biological ensemble classification model configuration 103). The biological ensemble classification model configuration comprises a biological classification ensemble model (e.g., biological classification ensemble model 200) comprising an unsupervised model (e.g., unsupervised model 202) and a supervised model (e.g., supervised model 204).

In various aspects, the unsupervised model is configured based on one or more of a principal component analysis (PCA), a Euclidean distance or correlation; a neighbor-based algorithm, a K-means algorithm, Quality Threshold (QT) algorithm, a Centroid algorithm, a Ward algorithm, or a Fuzzy C-Means clustering algorithm. For example, as described for FIG. 2A, an unsupervised model may comprise a PCA model comprising a reduced set of principal components. A configured model is a model that is trained on data (e.g., Raman-spectra data), and where the trained model may be used for predictive, classification, or otherwise product identification purposes thereafter. For example, in various aspects, the unsupervised model is trained with Raman-based spectra training data to configure the unsupervised model to output a first indicator (e.g., pass-fail indicator) of one or more biological product types.

In some aspects, an unsupervised model (e.g., unsupervised model 202) is configured to detect variability associated with identifying the one or more biological product types. For example, in some aspects, the variability comprises instrument (e.g., handheld analyzer) variability or sample lot-to-lot variability.

In additional aspects, unsupervised model (e.g., unsupervised model 202) may output an indicator (e.g., first indicated) based on whether the one or more biological product types satisfies a threshold value. As shown for FIG. 2A, the unsupervised model outputs a pass-fail determination based on the threshold value. The threshold value may be based on, or may be measured by, one or more of (but not limited to): a reduced Q-residual error, a Hotelling's T-squared value, a Mahalanobis distance value, or specific range values for principal component scores. For example, a biological ensemble classification model, of a configurable handheld biological analyzer (e.g., configurable handheld biological analyzer 102) may comprise a classification component selected to reduce a Q-residual error of the biological ensemble classification model. In this way, the biological classification mode is configured to identify the biological product type of a given biological product sample based on the classification component. Generally, Q-residuals are best used for biological products with single specification methods where lot-to-lot variability is the major source of variance among analyzers. Accordingly, as illustrated by FIG. 5A, Q-residuals may be used as a discriminating statistic to determine models (e.g., biological ensemble classification models as described herein) that are tolerant of analyzer-to-analyzer variability.

Additionally, or alternatively, Hotelling T² values may also be used with or instead of Q-residuals. Generally, Hotelling T² values represent a measure of the variation in each sample within a model (e.g., a biological ensemble classification model). Hotelling T² values indicate how far each sample is from a “center” (value of 0) of the model. Said another way, a Hotelling T² value is an indicator of distance from the model center. Distance from the center can often occur due to analyzer-to-analyzer variability. Using Hotelling T² values is advantageous to identify biological products with multiple specifications. In these cases, different concentrations of the active ingredient, excipients, etc., give rise to more substantial variability in the Raman spectra than lot-to-lot variation.

In the embodiment computer program listing of FIGS. 6A to 6C, at Code Section 7, a biological ensemble classification model configuration (e.g., biological ensemble classification model configuration 103) defines a set of PCA predictions specified for its biological ensemble classification model. Code Section 7 of FIGS. 6A to 6C also provides a script defining calculations for summary-of-fit statistic values (e.g., Hotelling T² values) and Q-residuals/values. Similarly, in the embodiment computer program listing of FIGS. 7A to 7C, at Code Section 7, a biological ensemble classification model configuration (e.g., biological ensemble classification model configuration 103) defines a set of PLSDA predictions specified for its biological ensemble classification model. Code Section 7 of FIGS. 7A to 7C also provides a script defining calculations for summary-of-fit statistic values. The script of Code Section 7, for each of FIGS. 6C and 7C, may be executed by first processor 110 as part of an ensemble model, including as for respective models (e.g., model 202m and 204, respectively) as described herein.

In various aspects, the supervised model (e.g., supervised model 204) may be trained with Raman-based spectra training data to configure the supervised model to output a second indicator of the one or more biological product types. The second indicator as output by the supervised model may be based on whether the one or more biological product types satisfies a biological product type prediction threshold value. For example, the supervised model may output a pass-fail determination based on the biological product type prediction threshold value, as described, for example, for FIG. 2A.

In various aspects, the supervised model is trained using one or more of a partial least squares discriminant analysis (PLSDA), a linear discriminant analysis (LDA), a K-nearest neighbor (KNN) algorithm, a soft independent modeling using class analogy (SIMCA), or a logistic regression discriminant analysis (LREGDA) algorithm. For example, as described herein for FIG. 2A, supervised model (e.g., supervised model 204) is a PLSDA model comprising comprises a reduced or otherwise optimized set of latent variables.

With further reference to FIG. 2B, at block 254, biological analytics method 250 further comprises receiving, at the first processor (e.g., first processor 110), a first Raman-based spectra dataset defining a first biological product sample as scanned by the first scanner.

At block 256, biological analytics method 250 further comprises executing, by the first processor (e.g., first processor 110), one or more spectral preprocessing algorithms as specified by the biological ensemble classification model configuration, to reduce a spectral variance of the first Raman-based spectra dataset. In various aspects, spectral variance refers to an analyzer-to-analyzer spectral variance between the first Raman-based spectra dataset and one or more other Raman-based spectra datasets of one or more corresponding other handheld biological analyzers. For example, spectral variance may exist between a Raman-based spectra dataset scanned by configurable handheld biological analyzer 102 and Raman-based spectra dataset scanned by configurable handheld biological analyzer 112. The spectral variance may exist even though each of the Raman-based spectra datasets, as scanned by each of the analyzers, is representative of the same biological product type. Such spectral variance can be caused by analyzer-to-analyzer variability and/or differences, such as software, having differences in versions, manufacture, age, operating environment (e.g., temperature), components, or other differences of Raman-based analyzers as described herein.

The spectral preprocessing algorithm is configured to reduce or otherwise mitigate the analyzer-to-analyzer spectral variance between the first Raman-based spectra dataset and the one or more other Raman-based spectra datasets. For example, in various embodiments, implementing or executing the spectral preprocessing algorithm (e.g., on first processor 110) minimizes statistical Type I (e.g., false positives) and/or Type II error (e.g., false negatives) associated with the identification of biological products (e.g., biological products 140). In various embodiments, the spectral preprocessing algorithm may reduce the analyzer-to-analyzer spectral variance among multiple configurable handheld biological analyzers (e.g., any of configurable handheld biological analyzers 102, 112, 114, and/or 116).

At block 258, biological analytics method 250 further comprises identifying or classifying, with the biological classification ensemble model (e.g., biological classification ensemble model 200), a biological product type based on the first Raman-based spectra dataset (e.g., the Raman-based spectra dataset as visualized and described for FIGS. 3A to 3C). For example, in various embodiments, once the execution sequence of a spectral preprocessing algorithm is executed (e.g., by first processor 110), e.g., as described herein with respect to FIGS. 3A to 3C and/or 6A to 6C or 7A to 7C, the preprocessed Raman-based datasets, e.g., aligned and/or normalized Raman-based spectra datasets (e.g., including Raman-based spectra datasets 322a, 322b, and 322c) as depicted in FIG. 3C, may be used by a configurable handheld biological analyzer (e.g., configurable handheld biological analyzer 102) to identify or classify biological products (e.g., biological products 140).

In various aspects herein, the biological ensemble classification model configuration may be transferred or otherwise deployed to another analyzer (e.g., a second configurable handheld biological analyzer). For example, with further reference to FIG. 2B, at block 260, biological analytics method 250 comprises transferring the biological ensemble classification model configuration to a second configurable handheld biological analyzer (e.g., configurable handheld biological analyzer 112).

At block 262, biological analytics method 250 comprises loading, into a second computer memory (e.g., of configurable handheld biological analyzer 112), the biological classification ensemble model configuration, the biological classification ensemble model configuration comprising the biological classification ensemble model.

At block 264, biological analytics method 250 further comprises receiving, by a second processor of the second configurable handheld biological analyzer (e.g., configurable handheld biological analyzer 112), a second Raman-based spectra dataset defining a second biological product sample as scanned by the second scanner.

At block 266, biological analytics method 250 further comprises identifying, by the second processor implementing the biological classification ensemble model (e.g., biological classification ensemble model 200), the biological product type based on the second Raman-based spectra dataset. The second biological product sample may comprise a new sample of the biological product type.

FIGS. 3A to 3C illustrate an example execution sequence of a spectral preprocessing algorithm of a configurable handheld biological analyzer (e.g., configurable handheld biological analyzer 102). Execution of the spectral preprocessing algorithm (e.g., by first processor 110) mitigates, reduces, or otherwise lessens the impact of differences unique to each analyzer (e.g., configurable handheld biological analyzers 102, 112, 114, and/or 116) and reduces variance among Raman-based spectra datasets produced by scans of those analyzers. One or more spectral preprocessing algorithms may be applied to modify and/or align Raman-based spectra data or otherwise information. In one aspect, a spectral preprocessing algorithm may comprise applying a derivative transformation to the first Raman-based spectra dataset to generate a modified Raman-based spectra dataset. In some aspects, the derivative transformation is applied to consecutive groups of 5 to 15 Raman intensity values across the Raman shift axis. It should be understood that additional and/or different ranges can be used, e.g., other ranges comprising 1 to 20 Raman intensity values. Further, in some aspects, the modified Raman-based spectra dataset may be centered.

In another aspect, a spectral preprocessing algorithm may further comprise aligning the modified Raman-based spectra dataset across a Raman shift axis. In some aspects, corresponding derivatives of the consecutive groups of 5 to 15 Raman intensity values are determined across the Raman shift axis.

In another aspect, a spectral preprocessing algorithm may further comprise normalizing the modified Raman-based spectra dataset across a Raman intensity axis.

In a still further aspects, the one or more spectral preprocessing algorithms may be executed to modify at least one of: (a) training data as used to train one or both of the supervised model or the unsupervised model; or (b) production data as used to produce an output from one or both of the supervised model or the unsupervised model.

FIG. 3A illustrates an example visualization 302 of example Raman-based spectra datasets (e.g., including Raman-based spectra datasets 302a, 302b, and 302c) as scanned by one or more handheld biological analyzers, in accordance with various embodiments disclosed herein. The Raman-based spectra datasets of FIG. 3A may comprise Raman-based spectra datasets (e.g., including Raman-based spectra datasets 302a, 302b, and 302c) used to generate a biological ensemble classification model configuration (e.g., biological ensemble classification model configuration 103) and its related biological ensemble classification model (e.g., biological ensemble classification model 200) as described herein. For example, the Raman-based spectra datasets of FIG. 3A may be those identified in Code Section 3 of FIG. 6A and/or FIG. 7A, as described herein.

In some embodiments, each of the Raman-based spectra datasets of FIG. 3A (e.g., including Raman-based spectra datasets 302a, 302b, and 302c) may represent scans by different configurable handheld biological analyzers (e.g., any of configurable handheld biological analyzers 102, 112, 114, and/or 116). In other embodiments, however, each of the Raman-based spectra datasets of FIG. 3A (e.g., including Raman-based spectra datasets 302a, 302b, and 302c) may represent multiple scans of the same configurable handheld biological analyzer (e.g., configurable handheld biological analyzers 102).

FIG. 3A depicts several Raman-based spectra datasets (e.g., including Raman-based spectra datasets 302a, 302b, and 302c), visualized across Raman intensity values (on Raman intensity axis 304) and light wavelength/frequency values (on Raman shift axis 306). Raman intensity axis 304 indicates the intensity of scattered light at a given wavelength across Raman shift axis 306. Raman intensity axis 304 can show many photons, as scanned by an analyzer (e.g., configurable handheld biological analyzer 102), are scattered by a biological product sample (e.g., where a data/value of 3 is a relative measure of intensity of the photons measured/scanned by first scanner 106). Raman shift axis 306 indicates the wavenumber (e.g., an inverse wavelength) of the scattered light. The units of wavenumbers (i.e., number of waves per centimeter (cm), cm⁻¹) provide an indication of the frequency or wavelength difference between the incident and scattered light. In the visualization 302 of FIG. 3A, shift axis 306 includes a range of 600 to 1500 cm⁻¹. Raman intensity axis 304 includes a Raman intensity range of 1 to 5. As shown in FIG. 3A, each of Raman-based spectra datasets (e.g., including Raman-based spectra datasets 302a Raman, 302b, and 302c) visualizes Raman intensity values measured across a light spectra range of 600 to 1500 cm⁻¹.

In addition, in various embodiments, each of the Raman-based spectra datasets of FIG. 3A (e.g., including Raman-based spectra datasets 302a, 302b, and 302c) may represent scans of the same biological product sample having the same biological product type. In such embodiments, as shown by FIG. 3A, even though any one or more of configurable handheld biological analyzer(s) may have scanned the same biological product sample having the same biological product type, variability exists in the Raman intensity values (on Raman intensity axis 304) of the Raman-based spectra datasets (e.g., including Raman-based spectra datasets 302a, 302b, and 302c) across the light wavelength/frequency values (on Raman shift axis 306). As described herein, the variability may have been caused by differences in software, manufacture, age, optical component(s), operating environment (e.g., temperature), or otherwise among the configurable handheld biological analyzers (e.g., any of configurable handheld biological analyzers 102, 112, 114, and/or 116).

FIG. 3B illustrates an example visualization 312 of modified Raman-based spectra datasets as modified from the Raman-based spectra datasets of FIG. 3A. For example, FIG. 3B may represent a first stage of an execution sequence of a spectral preprocessing algorithm. Visualization 312 of FIG. 3B includes the same Raman intensity axis 304 and Raman shift axis 306 as described herein for FIG. 3A. In the embodiment of FIG. 3B, a processor (e.g., first processor 110) applies a derivative transformation to the Raman-based spectra datasets of FIG. 3A (e.g., including Raman-based spectra datasets 302a, 302b, and 302c) to generate a modified Raman-based spectra datasets (e.g., including Raman-based spectra datasets 312a, 312b, and 312c) as depicted in FIG. 3B. Specifically, in the embodiment of FIG. 3B, a first derivative with 11 to 15 point data smoothing is applied (i.e., Raman weighted averages of consecutive groups of 11 to 15 Raman shift values are determined and then a first derivative transformation is applied to the groups). Said another way, the derivative transformation shown by FIG. 3B includes determining, by a processor (e.g., first processor 110), Raman weighted averages of consecutive groups of 11 to 15 Raman shift values (of Raman intensity axis 304) across the Raman shift axis 306, and then determining, by the processor (e.g., first processor 110) corresponding derivatives of those Raman weighted averages across Raman shift axis 306. Application of the derivative transformation mitigates impact of background curvature, e.g., due to Rayleigh scatter\rejection optics and/or other dispersive elements. This is shown graphically, by comparison of visualization 302 of FIG. 3A and visualization 312 of FIG. 3B, where the variance (e.g., vertical and/or horizontal variance) of Raman-based spectra datasets (e.g., including Raman-based spectra datasets 302a, 302b, and 302c, as shown in FIG. 3A) is removed or reduced to produce the less variable, modified Raman-based spectra datasets (e.g., including Raman-based spectra datasets 312a, 312b, and 312c) as depicted in FIG. 3B.

Application of the derivative transformation, as visualized by FIG. 3B, is further illustrated by computer program listing of FIGS. 6A to 6C and FIGS. 7A to 7C. For example, in the embodiment computer program listing of FIGS. 6A to 6C and FIGS. 7A to 7C, at Code Section 4, for each set of Figures respectively, the biological ensemble classification model configuration includes a script, which is executable by first processor 110 of configurable handheld biological analyzer 102, which applies the derivative transformation algorithm, as described for FIG. 3B herein. For example, the script for Code Section 4 of FIG. 6A may be executed to apply a derivative transformation algorithm for an unsupervised model (e.g., model 202m for FIG. 2A), while the script for Code Section 4 of FIG. 7A may be executed to apply a derivative transformation algorithm for a supervised model (e.g., model 204m for FIG. 2A). It is to be understood that the script may be the same script, where the same transformation is applied to the data, and such transformation may be executed a single time for data to be used across both models in order to, for example, reduce computing resource utilization. In the alternative, each script, as exemplified for FIGS. 6A and 7A, may be executed independently for each of the different models.

FIG. 3C illustrates an example visualization of normalized Raman-based spectra datasets as a normalized version of the modified Raman-based spectra datasets of FIG. 3B. For example, FIG. 3C may represent a next stage or stages of the execution sequence of a spectral preprocessing algorithm. Visualization 322 of FIG. 3C includes the same Raman intensity axis 304 and Raman shift axis 306 as described herein for FIGS. 3A and 3B. For example, in one embodiment, the modified Raman-based spectra datasets (e.g., including Raman-based spectra datasets 312a, 312b, and 312c) as depicted in FIG. 3B, are aligned, by a processor (e.g., first processor 110) across Raman shift axis 306 to produce aligned Raman-based spectra datasets (e.g., including Raman-based spectra datasets 322a, 322b, and 322c) as depicted in FIG. 3C. Such alignment applies a correction for subtle y-axis shifts (i.e., of Raman intensity axis 304) caused by analyzer-to-analyzer variance/differences as described herein. Application of an alignment algorithm, as visualized by FIG. 3C, is further illustrated by computer program listing of FIGS. 6A to 6C and 7A to 7C. For example, in the embodiment computer program listing of FIGS. 6A to 6C and FIGS. 7A to 7C, at Code Section 6, respectively, the biological ensemble classification model configuration (e.g., biological ensemble classification model configuration 103) includes a script, which is executable by first processor 110 of configurable handheld biological analyzer 102, applies a mean-centering algorithm that adjusts the alignment of the modified Raman-based spectra datasets (e.g., including Raman-based spectra datasets 312a, 312b, and 312c) as depicted in FIG. 3B to remove or reduce spectral variance (e.g., vertical and/or horizontal variance) of these modified Raman-based spectra datasets. This adjustment results in the aligned Raman-based spectra datasets (e.g., including Raman-based spectra datasets 322a, 322b, and 322c) as depicted in FIG. 3C. For example, the script for Code Section 6 of FIG. 6B may be executed to apply a mean-centering algorithm for an unsupervised model (e.g., model 202m for FIG. 2A), while the script for Code Section 6 of FIG. 7B may be executed to apply a mean-centering algorithm for a supervised model (e.g., model 204m for FIG. 2A). It is to be understood that the script may be the same script, where the same algorithm is applied to the data, and such algorithm may be executed a single time for data to be used across both models in order to, for example, reduce computing resource utilization. In the alternative, each script, as exemplified for FIGS. 6B and 7B, may be executed independently for each of the different models.

Additionally, or alternatively, in another embodiment, the modified Raman-based spectra datasets (e.g., including Raman-based spectra datasets 312a, 312b, and 312c) as depicted in FIG. 3B, are normalized, by a processor (e.g., first processor 110) across Raman intensity axis 304 to produce aligned Raman-based spectra datasets (e.g., including Raman-based spectra datasets 322a, 322b, and 322c) as depicted in FIG. 3C. Such normalization applies a robust normalization algorithm to account for intensity-axis variation (i.e., variations in intensity values across Raman intensity axis 304) caused by analyzer-to-analyzer variance/differences ad described herein. Application of a normalization algorithm, as visualized by FIG. 3C, is further illustrated by computer program listing of FIGS. 6A to 6C and FIGS. 7A to 7C. For example, in the embodiment computer program listing of FIGS. 6A to 6C and FIGS. 7A to 7C, at Code Section 5, respectively, the biological ensemble classification model configuration includes a script, which is executable by first processor 110 of configurable handheld biological analyzer 102, that applies a normalization algorithm that normalizes the modified Raman-based spectra datasets (e.g., including Raman-based spectra datasets 312a, 312b, and 312c) as depicted in FIG. 3B to remove or reduce spectral variance (e.g., vertical and/or horizontal variance) of these modified Raman-based spectra datasets. This normalization results in normalized Raman-based spectra datasets (e.g., including Raman-based spectra datasets 322a, 322b, and 322c) as depicted in FIG. 3C. In particular, in the embodiment of FIGS. 6A to 6C and FIGS. 7A to 7C, an standard normal variate (SNV) algorithm is applied, e.g., by first processor 110, to the modified Raman-based spectra datasets (e.g., including Raman-based spectra datasets 312a, 312b, and 312c) as depicted in FIG. 3B to produce aligned Raman-based spectra datasets (e.g., including Raman-based spectra datasets 322a, 322b, and 322c) as depicted in FIG. 3C. For example, the script for Code Section 5 of FIG. 6B may be executed to apply a SNV algorithm for an unsupervised model (e.g., model 202m for FIG. 2A), while the script for Code Section 5 of FIG. 7B may be executed to apply a SNV algorithm for a supervised model (e.g., model 204m for FIG. 2A). It is to be understood that the script may be the same script, where the same algorithm is applied to the data, and such algorithm may be executed a single time for data to be used across both models in order to, for example, reduce computing resource utilization. In the alternative, each script, as exemplified for FIGS. 6B and 7B, may be executed independently for each of the different models.

Application of the alignment and/or normalization algorithms (e.g., as described for FIG. 3C) removes or reduces spectral variance of the modified Raman-based spectra datasets (e.g., including Raman-based spectra datasets 312a, 312b, and 312c) as depicted in FIG. 3B. This is shown graphically, by comparison of visualization 312 of FIG. 3B and visualization 322 of FIG. 3C, where the spectral variance (e.g., vertical and/or horizontal variance) of Raman-based spectra datasets (e.g., including Raman-based spectra datasets 312a, 312b, and 312c, as shown in FIG. 3B) is removed or reduced to produce the less variable, aligned and/or normalized Raman-based spectra datasets (e.g., including Raman-based spectra datasets 322a, 322b, and 322c) as depicted in FIG. 3C.

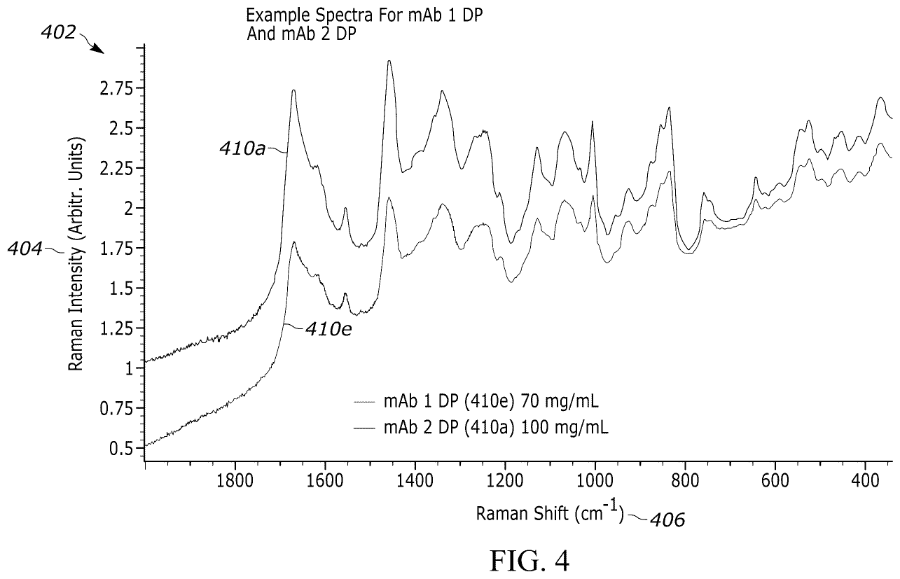

FIG. 4 illustrates an example visualization 402 of Raman-based spectra datasets for mAb 1 DP 410e and mAb 2 DP 410a as scanned by a handheld biological analyzer (e.g., configurable handheld biological analyzer 102), in accordance with various embodiments disclosed herein. The Raman-based spectra datasets (e.g., including Raman-based spectra datasets 410a and 410e) are visualized across Raman intensity values (on Raman intensity axis 404) and light wavelength/frequency values (on Raman shift axis 406). Raman intensity axis 404 indicates the intensity of scattered light at a given wavelength across Raman shift axis 406. Raman intensity axis 404 can show many photons, as scanned by an analyzer (e.g., configurable handheld biological analyzer 102), as scattered by a biological product sample (e.g., where a data/value of 3 is a relative measure of intensity of the photons measured/scanned by first scanner 106). Raman shift axis 406 indicates the wavenumber (e.g., an inverse wavelength) of the scattered light. The units of wavenumbers (i.e., number of waves per centimeter (cm), cm⁻¹) provide an indication of the frequency or wavelength difference between the incident and scattered light. In the visualization 402 of FIG. 4, shift axis 406 includes a range of 350 to 2000 cm⁻¹. Raman intensity axis 404 includes a Raman intensity range of 0 to 3. As shown in FIG. 4, each of Raman-based spectra datasets (e.g., including Raman-based spectra datasets 410a and 410e) visualizes Raman intensity values measured across a light spectra range of 600 to 1500 cm⁻¹.

The visualization of FIG. 4 represents Raman-based spectra having similar datasets and are therefore can be difficult to distinguish. That is, in the visualization of example of FIG. 4, the Raman spectrum (Raman Intensity across Raman Shift) for mAb 1 DP 410e and mAb 2 DP 410a makes it difficult a configurable handheld biological analyzer 102 to distinguish. However, it should be noted that different product types have resulting Raman spectra that are not exactly the same (but they can be similar). For example, such different (but similar) Raman spectra, such as mAb 1 DP 410e and mAb 2 DP 410a as shown for FIG. 4, can have tracking and/or overlapping Raman spectral features, but where at least some Raman spectral features are different. Thus, in various aspects herein, a first biological product type of a given one or more biological product types and a second biological product type of the given one or more biological product types may have similar Raman-based spectra (e.g., mAb 1 DP 410e and mAb 2 DP 410a as shown for FIG. 4), but where such spectra have different features discoverable by training a model, e.g., a supervised or unsupervised model.

FIG. 5A illustrates an example visualization 502 of Q-residual error of a non-ensemble unsupervised biological classification model when the Raman-based spectra datasets, including those of FIG. 4, are provided as input. At least in some aspects, FIG. 5A represents a non-ensemble unsupervised model demonstrating a model having a best-case scenario and showing that it is not possible to obtain true positive (e.g., a true negative) having 100% predictive ability or otherwise accuracy. In particular, as shown for key 509, mAb 1 DP 410e is represented as mAb 1-TM5137 for mAb 1 DP 410e as scanned by a scanner designated as TM5137 (510mAb15137 of visualization 502). mAb 2 DB 410a is represented as mAb 2-TM5137 for mAb 2 DP 410a as scanned by the scanner designed as TM5137 (510mAb25137 of visualization 502). Additional Raman-based spectra datasets (not shown for FIG. 4) as scanned by different configurable handheld biological analyzers are also shown. The configurable handheld biological analyzers, which there are three in the example of FIG. 5A, are identified or otherwise indicated as follows: TM4164, TM5133, TM5137. Thus, the various Raman-based spectra datasets as scanned by the various scanner further include mAb 1 datasets mAb1-TM4164 (510mAb14164 of visualization 502) and mAb 1-TM5133 (510mAb15133 of visualization 502), as well as mAb 2 datasets mAb 2-TM4164 (510mAb24164 of visualization 502) and mAb 2-TM5133 (510mAb25133 of visualization 502). Key 509 also indicates Q-residual threshold 508, which is a threshold value of FIG. 5A used to distinguish between mAb 1 and mAb 2.

For example, visualization 502 may include classification results of a PCA model alone, where mAb 1 DP 410e is treated as a target product. In the example of FIG. 5A, visualization 502 includes a Q-residual error axis 504 and Linear Index axis 506. Linear Index 506 represents the ordering of individual spectra within the dataset used to evaluate model performance. Q-residuals (e.g., of Q-residual error axis 504) represents error values across a range of 0 to 1.5 and provide a lack-of-fit statistic calculated as the sum of squares of each product sample. Q-residuals represent a magnitude of variation remaining in each sample after projection through a given model (e.g., a PCA model of a biological ensemble classification model as described herein). More generally, as illustrated by the embodiment of FIG. 5A, Q-residual values (along Q-residual error axis 504) serve as a discriminating statistic. Q-residuals is a measure of what is not explained, by a given model. For example, in an embodiment where a PCA model is used (e.g., where a spectrum is projected on a first principal component), the values of FIG. 5A would show what is left (the residuals) after the scanned data (e.g., 410a and 410e) is projected onto the first principal component.

Visualization 502 illustrates a limitation of using an PCA model alone based on reduced Q-Residuals alone as a distinguishing feature. For example, as illustrated for FIG. 5A, a PCA model (or other classification model) alone can be inaccurate, where, for example, FIG. 5A shows Q-Residuals for a single PCA model calibrated for mAb 1 DP. In the example of FIG. 5A, such model is not sensitive enough to distinguish from mAb 1 DP from mAb 2 DP.

More specifically, in the example of FIG. 5A, a single PCA model was trained or otherwise configured using mAb 1 DP spectra for two samples/lots acquired on two Raman instruments (TM5133 and TM5137). The model was built using standard preprocessing parameters (e.g., Truncate spectral range to 350-1801 cm⁻¹, then take a first derivative Savitsky-Golay smoothing, apply a standard normal variate, and then mean center the values). Model validation was performed using independent spectra for three lots each of mAb 1 DP and mAb 2 DP, acquired on three Raman instruments, e.g., configurable handheld biological analyzers (TM4164, TM5133, and TM5137). In some aspects, elevated residuals are typically observed on instruments not included in the model build (i.e., network instruments), and so the pass/fail criterion is adjusted to compensate. In the example of FIG. 5A, the Raman profile for mAb 2 DP is similar enough to mAb 1 DP such that the two products cannot be distinguished based on their Q-residual values within the normal/expected instrument variability for any of the configurable handheld biological analyzers.

FIG. 5B illustrates an example visualization 552 of predictive output of a supervised biological classification ensemble model (e.g., biological classification ensemble model 200), in accordance with various embodiments herein, when the Raman-based spectra datasets, including those of FIG. 4, are provided as input. In at least some aspects, FIG. 5B represents a non-ensemble supervised model that provides a true positive rate (i.e., a true negative rate) at 100% prediction or otherwise accuracy for distinguishing mAb 1 DP and mAb 2 DP. This model allows for fine tuning of the unsupervised model (e.g., as described for FIG. 5A) such that the true positive rate is 100% (or an appropriate value thereof). In addition, the second, supervised model as described for FIG. 5B provides an improved performance over the model of FIG. 5A alone to achieve true negative rate (e.g., 100% or approximate value thereof). In the example of FIG. 5B, an ensemble model is used, as described herein, for example, biological classification ensemble model 200, where each of an unsupervised model and supervised model is used. By way of example, FIG. 5B illustrates use of an PLSDA model where mAb 1 DP treated as the target product. In the example of FIG. 5B, visualization 552 includes a Y-Predicted axis 554 and Linear Index axis 556. Linear Index 556 represents the ordering of individual spectra within the dataset across a range of 0 to 275. Y-Predicted axis 554 represents a range of values that represents a label output or otherwise dependent variable output of the PLSDA model with respect to mAb 1 DP 70 mg/mL. In various aspects, FIG. 5B demonstrates output of the second PLSDA model (e.g., model 204m as shown for FIG. 2A). For the precursor PCA model (e.g., model 202m of FIG. 2A), conditions are adjusted (e.g., where the Q-residuals Pass/Fail threshold is set) so that the true positive rate can be 100%. While the PCA model may generate false positive results for, e.g., mAb 2, the supervised biological classification ensemble model can avoid false positives, having a true positive rate of 100%.

Key 560 includes the same values with respect to Raman-based spectra datasets and related configurable handheld biological analyzers, including mAb 1 DP 410e is represented as mAb1-TM5137 for mAb 1 DP 410e as scanned by a scanner designated as TM5137 (i.e., 510mab15137 of visualization 502), and so forth as described for FIG. 5A. However, Key 560 also includes Y-Discriminant threshold 558, which is a threshold value of FIG. 5B used to distinguish between mAb 1 and mAb 2. Instead of using a Q-residual threshold (as for FIG. 5A), the model of FIG. 5A uses the output of PLSDA, which is a predictive value that can range, in the example of FIG. 5B, from −0.1 to 1.2 with respect to mAb 1 DP 70 mg/mL. In this example, a nominal value of 1 corresponds to a perfect fit with class mAb 1 DP 70 mg/mL, and a nominal value of 0 corresponds to a perfect fit with class mAb 2 DP 100 mg/mL.

Visualization 552 of FIG. 5B illustrates a new, and accurate, output of a biological classification ensemble model (e.g., biological classification ensemble model 200), that shows a prediction that identifies mAb 1 when the output value is above the threshold (e.g., Y-Discriminant threshold 558), and that identifies mAb 2 when the output value is below the threshold (e.g., Y-Discriminant threshold 558), thereby allowing for accurate classification of mAb 1 DP and mAb 2 DP. In some aspects, for example, as additionally described for FIG. 2A, the output value may be used to “PASS” a product (e.g., when the output value is above approximately 0.5 as shown for FIG. 5B). Similarly, the output value may be used to “FAIL” a product (e.g., when the output value is below approximately 0.5 as shown for FIG. 5B).

In the example of FIG. 5B, a model (e.g., a PLSDA model) was trained or otherwise configured using mAb 1 DP and mAb 2 DP spectra (two samples/lots each using two Raman instruments, TM5133 and TM5137). The model was built using standard preprocessing parameters used for the PCA model of FIG. 5A (e.g., Truncate spectral range to 350-1801 cm⁻¹, then take a first derivative Savitsky-Golay smoothing, apply a standard normal variate, and then mean center the values). Model validation was performed using independent spectra for three lots each of mAb 1 DP and mAb 2 DP, acquired on three Raman instruments (TM4164, TM5133, and TM5137). As shown for FIG. 5B, the ensemble model comprising the supervised PLSDA algorithm is better able (compared to the PCA model alone) to distinguish mAb 1 DP and mAb 2 DP based on their characteristic Raman spectra, mitigating the impact of instrument-to-instrument variation. In this way, a supervised model of a biological classification ensemble model (e.g., biological classification ensemble model 200) may be configured to distinguish a biological product sample having a given biological product type from a different biological product sample having a different (but similar) biological product type (e.g., as is the case with mAb 1 DP and mAb 2 DP). In various cases, a first biological product type and the different biological product type may each have distinct localized features within a similar Raman spectra range, and where the biological classification ensemble model 200 is able to use such features to distinguish between the first biological product type and the different biological product type.

In this way, the ensemble model (e.g., biological classification ensemble model 200) is able to leverage the advantages of both a classification and predictive model (e.g., an unsupervised model and supervised model) in order to increase accuracy of identification and detection of a configurable handheld biological analyzer 102 to distinguish product types. By contrast, a single model (e.g., PCA model) that is broadly specific against other products with dissimilar or moderately similar Raman profiles can experience difficulty in detecting product types having similar Raman spectra datasets or otherwise Raman related features. One such approach is described by publication WO 2021/081263, titled “Configurable Handheld Biological Analyzers for Identification of Biological Products based on Raman Spectroscopy”, and filed as PCT/US2020/056961 on Oct. 23, 2020. While the single model provides an improvement over conventional detection methods, the biological classification ensemble model (e.g., biological classification ensemble model 200) as described herein allows for further accurate identification and detection of product types. Generally, the biological classification ensemble model is configured to increase accuracy and product type identification performance by chaining or otherwise combining the predictions from two or more artificial intelligence models. For example, a first model may comprise an unsupervised model as described for FIG. 2A. The first model may comprise, by way of non-limiting example, a PCA model. A PCA model provides a first layer of specificity and identification among different product types. The PCA model is also unsupervised, meaning that it does not need to be trained to differentiate among product samples. However, the PCA model cannot, in some cases, distinguish product types based on Raman spectra with a sufficient degree of certainty.

To improve upon the PCA model, the ensemble model (e.g., biological classification ensemble model 200) as described herein may be developed where the addition of a supervised model, or otherwise second model, is added to address product types having similar Raman spectra. The second model may comprise a PLSDA model as described, for example, for FIG. 2A herein. A PLSDA model can be trained to have a high degree of specificity with respect to product types (e.g., product types that are different, but have similar Raman spectra, such as mAb 1 and mAb 2).

It should be noted that a typical analyzer (not implementing or executing biological ensemble classification model configuration 103 as described herein) generally produces significant numbers of Type I (e.g., false positives) and Type II errors (e.g., false negatives) when attempting to identify, measure, or classify such biological product types.

However, a configurable handheld biological analyzer (e.g., configurable handheld biological analyzer 102), loaded and executing a biological ensemble classification model configuration (e.g., biological ensemble classification model configuration 103) as described herein, may be used to accurately identify, classify, measure, or otherwise distinguish the biological product types of mAb 2 DS/DP, mAb 1 DP, mAb 3 DP, among others. In particular, across the range of Raman-spectra, each product type may be varying localized features that have different Raman intensity values (having different shapes, peaks, or otherwise distinct/different relative intensities), that are specific to each of biological product types, e.g., mAb 2 DS/DP, mAb 1 DP, mAb 3 DP, etc. Because of this, the distinct localized features provide a source of product specific information that can be used by configurable handheld biological analyzer 102 to identify, classify, or otherwise distinguish biological products as described herein. It should be understood, however, that these biological product types are merely examples, and that other biological product types or biological products may be identified, classified, measured, or otherwise distinguished in a same or similar manner as described for the various embodiments herein.

As described herein, a biological ensemble classification model (e.g., biological ensemble classification model 200) may be configured, to identify, classify, measure, or otherwise distinguish a given biological product sample having a given biological product type (e.g., mAb 1 DP) from a different or second biological product sample having a different or second biological product type (e.g., mAb 2 DS/DP). For example, as described herein, configurable handheld biological analyzer 102, once configured with biological ensemble classification model configuration 103, can execute a spectral preprocessing algorithm (e.g., as described herein FIGS. 3A to 3C) on a Raman-based spectra dataset as received by first scanner 106. Once the Raman-based spectra dataset is preprocessed by the spectral preprocessing algorithm, the configurable handheld biological analyzer 102 may identify or classify a biological product using an ensemble model (e.g., as described for FIGS. 2A and 2B).

### Additional Examples

The below additional examples provide additional support in accordance with various embodiments described herein. In particular, the below additional examples demonstrate Raman spectroscopy for rapid identity (ID) verification of biotherapeutic protein products in solution. The examples demonstrate a unique combination of Raman features associated with both a therapeutic agent and excipients as the basis for product differentiation. Product ID methods (e.g., biological analytics methods), as described herein, include acquiring Raman spectra of the target product(s) on multiple Raman analyzers (e.g., configurable handheld biological analyzers, as described herein). The spectra may then subjected to dimension reduction using principal component analysis (PCA) and/or partial least squares discriminant analysis (PLSDA) to define product-specific models (e.g., biological classification ensemble models) which serve as the basis for an product ID determination for configurable handheld biological analyzers and biological analytics method for identification of biological products based on Raman spectroscopy as described herein. The product-specific models (e.g., biological classification ensemble models) can be transferred to separate instruments (e.g., configurable handheld biological analyzers) that are validated for product testing. These may be used for various purposes including quality control, incoming quality assurance, and manufacturing. Such analyzers and methods may be used across different Raman apparatuses (e.g., configurable handheld biological analyzers) from different manufacturers. In this way, the additional examples further demonstrate that the Raman ID analyzers and methods describe herein (e.g., the configurable handheld biological analyzers and related methods) provide various uses and tests for solution-based protein products in the biopharmaceutical industry.

### Additional Examples—Raman Instrumentation (e.g., Configurable Handheld Biological Analyzers) and Measurements

With respect to the additional examples, Raman spectra were measured using configurable handheld biological analyzers, as described herein. For example, in certain embodiments, configurable handheld biological analyzers may be a Raman-based handheld analyzer, such as a TruScan™ RM Handheld Raman Analyzer as provided by Thermo Fisher Scientific Inc. In such embodiments, the configurable handheld biological analyzer may implement TruTools™ chemometrics software package. Although, it is to be understood, that other brands or types of Raman analyzers using additional and/or different software packages may be used in accordance with the disclosure herein. In some embodiments, the configurable handheld biological analyzers may be configured with a 785 nm grating-stabilized laser source (250 mW maximum output) coupled with focusing optics (e.g., 0.33 NA, 18 mm working distance, >0.2 mm spot) for sample interrogation. For the additional examples, product solutions, contained in glass vials, were secured in front of the focusing optics using a vial adapter for the configurable handheld biological analyzers. All spectra were collected using the following, identical spectral acquisition settings (although other settings may be used), e.g., laser power=250 mW, integration time=1000 ms, number of spectral co-additions=70. For the additional examples, product spectra were collected over a period of time using three different configurable handheld biological analyzers (hereafter referred to as configurable handheld biological analyzers 1-3) and/or instruments dedicated to the configuration and/or development of biological analytics method(s) for identification of biological products based on Raman spectroscopy as described herein. It is to be understood that additional or fewer analyzers using the same or different settings may be used for setting, configuring, or otherwise initializing configurable handheld biological analyzers, and the related biological analytics method(s), as described herein.

### Additional Examples—Development of Multivariate Raman ID Biological Analytics Methods

Raman spectral models (e.g., biological classification ensemble models) may be generated, developed, or loaded as described herein. For example, in some embodiments, SOLO software equipped with a Model Exporter add-on (Solo+Model_Exporter version 8.2.1; Eigenvector Research, Inc.) may be used to generate, develop, or load a Raman spectral models (e.g., biological classification ensemble models). It is to be understood, however, that other software may be used to generate, develop, or load a Raman spectral models (e.g., biological classification ensemble models). Spectra used to build models may generally be collected as replicate scans on two or more distinct lots of material using configurable handheld biological analyzers (e.g., three configurable handheld biological analyzers). The spectra is generally acquired over multiple days for the purpose of including instrument drift. In some embodiments, prior to incorporation into a model (e.g., biological classification ensemble model), the spectral range may be reduced to exclude detector noise at >1800 cm-1 and background variability arising from the Rayleigh line-rejection optics at <400 cm-1. The spectra may be further preprocessed and mean-centered, as described herein, for each model. The models additionally may be refined by cross-validation, using a random subset procedure, by reference to the Raman spectra of the target and challenge products, as shown in Table 1.

The biological ensemble classification model configuration (e.g., a PCA and PLSDA ensemble model configuration), along with the Raman spectral acquisition parameters, may be configured or loaded configurable handheld biological analyzers and/or use biological analytics method(s) for identification of biological products based on Raman spectroscopy as described herein. The acceptance (e.g., pass-fail) criteria for each method may also be specified.

As described herein, the pass-fail criteria for unsupervised models may be based on threshold values for reduced Hotelling's T² (Tᵣ²) and Q-residuals (Qᵣ), which are two summary statistics that generally describe how well a Raman spectrum is described by a biological ensemble classification model (e.g., PCA model). Equations (1)-(4) below provide example user-selectable decision logic options for a positive identification or determination (e.g., pass-fail criteria) by an unsupervised model (e.g., PCA model) of a biological ensemble classification model (e.g., biological ensemble classification model 200):

$${Q}_{r} \leq 1.000000 \qquad ( 1 )$$

$${T}_{r}^{2} \leq 1 \text{.000000} \qquad ( 2 )$$

$${Q}_{r} + {T}_{r}^{2} \leq 1.000 \; 000 \qquad ( 3 )$$

$$\sqrt{{[ {Q}_{r} ]}^{2} + {[ {T}_{r}^{2} ]}^{2}} \leq 1. \qquad ( 4 )$$

In the above example equations, the Hotelling's T² and Q-residuals values are normalized (i.e., reduced, Tᵣ² and Qᵣ, respectively) by dividing the original values by the corresponding confidence interval, thereby setting the value of the upper bound to a value of 1. It should be understood that different and/or additional equations, specifying different and/or additional threshold values, may be used by the configurable handheld biological analyzers and/or use biological analytics method(s) without departing from the disclosure herein.

### Additional Description

The above description herein describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with, or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 146B7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF: cMet axis (HGF/SF: c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4ß7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Humatrope® (somatropin, Human Growth Hormone); Humira® (adalimumab); Vectibix® (panitumumab), Xgeva® (denosumab), Prolia® (denosumab), Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP IIb/IIia receptor monoclonal antibody); Actemra® (anti-IL6 Receptor mAb); Avastin® (bevacizumab), HuMax-CD4 (zanolimumab); Rituxan® (rituximab, anti-CD20 mAb); Tarceva® (erlotinib); Roferon-A®-(interferon alfa-2a); Simulect®(basiliximab); Prexige® (lumiracoxib); Synagis® (palivizumab); 146B7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Ra mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-1103); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGB mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFß mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, or BPS 804 (Novartis) and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. Antagonistic antibodies for human calcitonin gene-related peptide (CGRP) receptor such as but not limited to mAb 1 and bispecific antibody molecules that target the CGRP receptor and other headache targets may also be delivered with a drug delivery device of the present disclosure. Additionally, bispecific T cell engager (BiTE®) molecules such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

### Additional Considerations

Although the disclosure herein sets forth a detailed description of numerous different embodiments, it should be understood that the legal scope of the description is defined by the words of the claims set forth at the end of this patent and equivalents. The detailed description is to be construed as exemplary only and does not describe every possible embodiment since describing every possible embodiment would be impractical. Numerous alternative embodiments may be implemented, using either current technology or technology developed after the filing date of this patent, which would still fall within the scope of the claims.

The following additional considerations apply to the foregoing discussion. Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

The term “coupled to” used herein does not require a direct coupling or connection, such that two items may be “coupled to” one another through one or more intermediary components or other elements, such as an electronic bus, electrical wiring, mechanical component, or other such indirect connection.

Hardware modules may provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and may operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location, while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other embodiments, the one or more processors or processor-implemented modules may be distributed across a number of geographic locations.

This detailed description is to be construed as exemplary only and does not describe every possible embodiment, as describing every possible embodiment would be impractical, if not impossible. A person of ordinary skill in the art may implement numerous alternate embodiments, using either current technology or technology developed after the filing date of this application.

Those of ordinary skill in the art will recognize that a wide variety of modifications, alterations, and combinations can be made with respect to the above-described embodiments without departing from the scope of the invention, and that such modifications, alterations, and combinations are to be viewed as being within the ambit of the inventive concept.

The patent claims at the end of this patent application are not intended to be construed under 35 U.S.C. § 112 (f) unless traditional means-plus-function language is expressly recited, such as “means for” or “step for” language being explicitly recited in the claim(s). The systems and methods described herein are directed to an improvement to computer functionality and improve the functioning of conventional computers.

### Aspects of the Disclosure

Aspect 1. A configurable handheld biological analyzer for identification of biological products based on Raman spectroscopy using ensemble artificial intelligence (AI), the configurable handheld biological analyzer comprising: a first housing adapted for handheld manipulation; a first scanner carried by the first housing; a first processor communicatively coupled to the first scanner; and a first computer memory communicatively coupled to the first processor, wherein the first computer memory is configured to load a biological ensemble classification model configuration, the biological ensemble classification model configuration comprising a biological classification ensemble model comprising an unsupervised model and a supervised model, wherein the unsupervised model is trained with Raman-based spectra training data to configure the unsupervised model to output a first indicator of one or more biological product types, wherein the supervised model is trained with Raman-based spectra training data to configure the supervised model to output a second indicator of the one or more biological product types, wherein the biological classification ensemble model configuration further comprises one or more spectral preprocessing algorithms, the first processor configured to execute the one or more spectral preprocessing algorithms to reduce a spectral variance of a first Raman-based spectra dataset when the first Raman-based spectra dataset is received by the first processor, and wherein the biological classification ensemble model is configured to execute on the first processor, the first processor configured to (1) receive a first Raman-based spectra dataset defining a first biological product sample as scanned by the first scanner, and (2) identify, with the biological classification ensemble model, a biological product type of the one or more biological product types based on the first Raman-based spectra dataset.

Aspect 2. The configurable handheld biological analyzer of aspect 1, wherein the biological ensemble classification model configuration is electronically transferrable to a second configurable handheld biological analyzer, the second configurable handheld biological analyzer comprising: a second housing adapted for handheld manipulation; a second scanner coupled to the second housing; a second processor communicatively coupled to the second scanner; and a second computer memory communicatively coupled to the second processor, wherein the second computer memory is configured to load the biological classification ensemble model configuration, the biological classification ensemble model configuration comprising the biological classification ensemble model, wherein the biological classification ensemble model is configured to execute on the second processor, the second processor configured to (1) receive a second Raman-based spectra dataset defining a second biological product sample as scanned by the second scanner, and (2) identify, with the biological classification ensemble model, the biological product type based on the second Raman-based spectra dataset, wherein the second biological product sample is a new sample of the biological product type.

Aspect 3. The configurable handheld biological analyzer of aspect 1, wherein the spectral variance is an analyzer-to-analyzer spectral variance between the first Raman-based spectra dataset and one or more other Raman-based spectra datasets of one or more corresponding other handheld biological analyzers, each of the one or more other Raman-based spectra datasets representative of the biological product type, and wherein the one or more spectral preprocessing algorithms are configured to mitigate the analyzer-to-analyzer spectral variance between the first Raman-based spectra dataset and the one or more other Raman-based spectra datasets.

Aspect 4. The configurable handheld biological analyzer of aspect 3, wherein the one or more spectral preprocessing algorithms comprises: applying a derivative transformation to the first Raman-based spectra dataset to generate a modified Raman-based spectra dataset, aligning the modified Raman-based spectra dataset across a Raman shift axis, and normalizing the modified Raman-based spectra dataset across a Raman intensity axis.

Aspect 5. The configurable handheld biological analyzer of aspect 4, wherein the modified Raman-based spectra dataset is centered.

Aspect 6. The configurable handheld biological analyzer of aspect 4, wherein the derivative transformation is applied to consecutive groups of 5 to 15 Raman intensity values across the Raman shift axis.

Aspect 7. The configurable handheld biological analyzer of aspect 5, wherein corresponding derivatives of the consecutive groups of 5 to 15 Raman intensity values are determined across the Raman shift axis.

Aspect 8. The configurable handheld biological analyzer of any one of aspects 1-7, wherein the unsupervised model is configured to detect variability associated with identifying the one or more biological product types.

Aspect 9. The configurable handheld biological analyzer of aspect 8, wherein the variability comprises instrument variability or sample lot-to-lot variability.

Aspect 10. The configurable handheld biological analyzer of any one of aspects 1-9, wherein the biological classification ensemble model identifies the biological product type upon determination that the first indicator passes a first pass-fail based threshold value and that the second indicator passes a second pass-fail based threshold value.

Aspect 11. The configurable handheld biological analyzer of any one of aspects 1-9, wherein the first indicator as output by the unsupervised model is based on whether the one or more biological product types satisfies a threshold value.

Aspect 12. The configurable handheld biological analyzer of aspect 11, wherein the unsupervised model outputs a pass-fail determination based on the threshold value.

Aspect 13. The configurable handheld biological analyzer of aspect 11 or 12, wherein the threshold value is based on one or more of: a reduced Q-residual error, a Hotelling's T-squared value, a Mahalanobis distance value, or specific range values for principal component scores.

Aspect 14. The configurable handheld biological analyzer of any one of aspects 1-13, wherein a first biological product type of the one or more biological product types and a second biological product type of the one or more biological product types have similar Raman-based spectra.

Aspect 15. The configurable handheld biological analyzer of any one of aspect 1-14, wherein the second indicator as output by the supervised model is based on whether the one or more biological product types satisfies a biological product type prediction threshold value.

Aspect 16. The configurable handheld biological analyzer of aspect 15, wherein the supervised model outputs a pass-fail determination based on the biological product type prediction threshold value.

Aspect 17. The configurable handheld biological analyzer of any one of aspects 1-16, wherein the computer memory is configured to load a new biological classification ensemble model, the new biological ensemble classification model comprising an updated unsupervised model and/or an updated supervised model.

Aspect 18. The configurable handheld biological analyzer of any one of aspects 1-17, wherein the biological classification ensemble model configuration is implemented in an extensible markup language (XML) format.

Aspect 19. The configurable handheld biological analyzer of any one of aspects 1-18, wherein the biological product type is of a therapeutic product.

Aspect 20. The configurable handheld biological analyzer of any one of aspects 1-19, wherein the biological product type is identified by the biological classification ensemble model during manufacture of a biological product having the biological product type.

Aspect 21. The configurable handheld biological analyzer of any one of aspects 1-20, wherein the supervised model of the biological classification ensemble model is configured to distinguish the first biological product sample having the biological product type from a different biological product sample having a different biological product type.

Aspect 22. The configurable handheld biological analyzer of aspect 21 wherein the biological product type and the different biological product type each have distinct localized features within a similar Raman spectra range.

Aspect 23. The configurable handheld biological analyzer of any one of aspects 1-22, wherein the biological classification ensemble model is generated by a remote processor being remote to the configurable handheld biological analyzer.

Aspect 24. The configurable handheld biological analyzer of any one of aspects 1-23, wherein the unsupervised model is configured based on: a principal component analysis (PCA), a Euclidean distance or correlation; a neighbor-based algorithm, a K-means algorithm, Quality Threshold (QT) algorithm, a Centroid algorithm, a Ward algorithm, or a Fuzzy C-Means clustering algorithm.

Aspect 25. The configurable handheld biological analyzer of aspect 24, wherein the unsupervised model is a PCA model, and wherein the PCA model comprises a reduced set of principal components.

Aspect 26. The configurable handheld biological analyzer of any one of aspects 1-25, wherein the supervised model is trained using a partial least squares discriminant analysis (PLSDA), a linear discriminant analysis (LDA), a K-nearest neighbor (KNN) algorithm, a soft independent modeling using class analogy (SIMCA), or a logistic regression discriminant analysis (LREGDA) algorithm.

Aspect 27. The configurable handheld biological analyzer of aspect 26, wherein the supervised model is a PLSDA model, and wherein the PLSDA model comprises a reduced set of latent variables.

Aspect 28. The configurable handheld biological analyzer of any one of aspects 1-27, wherein the unsupervised model is configured based on a principal component analysis (PCA) and the supervised model is configured on a partial least squares discriminant analysis (PLSDA).

Aspect 29. The configurable handheld biological analyzer of any one of aspects 1-28, wherein the one or more spectral preprocessing algorithms are executed to modify at least one of: (a) training data as used to train one or both of the supervised model or the unsupervised model; or (b) production data as used to produce an output from one or both of the supervised model or the unsupervised model.

Aspect 30. A biological analytics method for identification of biological products based on Raman spectroscopy using ensemble artificial intelligence (AI), the biological analytics method comprising: loading, into a first computer memory of a first configurable handheld biological analyzer having a first processor and a first scanner, a biological ensemble classification model configuration, the biological ensemble classification model configuration comprising a biological classification ensemble model comprising an unsupervised model and a supervised model, wherein the unsupervised model is trained with Raman-based spectra training data to configure the unsupervised model to output a first indicator of one or more biological product types, and wherein the supervised model is trained with Raman-based spectra training data to configure the supervised model to output a second indicator of the one or more biological product types; receiving, at the first processor, a first Raman-based spectra dataset defining a first biological product sample as scanned by the first scanner; executing, by the first processor, one or more spectral preprocessing algorithms as specified by the biological ensemble classification model configuration, to reduce a spectral variance of the first Raman-based spectra dataset; and identifying, with the biological classification ensemble model, a biological product type based on the first Raman-based spectra dataset.

Aspect 31. The biological analytics method of aspect 30 further comprising: transferring the biological ensemble classification model configuration to a second configurable handheld biological analyzer; loading, into a second computer memory, the biological classification ensemble model configuration, the biological classification ensemble model configuration comprising the biological classification ensemble model; receiving, by a second processor of the second configurable handheld biological analyzer, a second Raman-based spectra dataset defining a second biological product sample as scanned by the second scanner; and identifying, by the second processor implementing the biological classification ensemble model, the biological product type based on the second Raman-based spectra dataset, wherein the second biological product sample is a new sample of the biological product type.

Aspect 32. The biological analytics method of aspect 30, wherein the spectral variance is an analyzer-to-analyzer spectral variance between the first Raman-based spectra dataset and one or more other Raman-based spectra datasets of one or more corresponding other handheld biological analyzers, each of the one or more other Raman-based spectra datasets representative of the biological product type, and wherein the one or more spectral preprocessing algorithms are configured to mitigate the analyzer-to-analyzer spectral variance between the first Raman-based spectra dataset and the one or more other Raman-based spectra datasets.

Aspect 33. The biological analytics method of aspect 32, wherein the one or more spectral preprocessing algorithms comprises: applying a derivative transformation to the first Raman-based spectra dataset to generate a modified Raman-based spectra dataset, aligning the modified Raman-based spectra dataset across a Raman shift axis, and normalizing the modified Raman-based spectra dataset across a Raman intensity axis.

Aspect 34. The biological analytics method of aspect 33, wherein the modified Raman-based spectra dataset is centered.

Aspect 35. The biological analytics method of aspect 33, wherein the derivative transformation is applied to consecutive groups of 5 to 15 Raman intensity values across the Raman shift axis.

Aspect 36. The biological analytics method of aspect 35, wherein corresponding derivatives of the consecutive groups of 5 to 15 Raman intensity values are determined across the Raman shift axis.

Aspect 37. The biological analytics method of any one of aspects 30-36, wherein the unsupervised model is configured to detect variability associated with identifying the one or more biological product types.

Aspect 38. The biological analytics method of aspect 37, wherein the variability comprises instrument variability or sample lot-to-lot variability.

Aspect 39. The biological analytics method of any one of aspects 30-38, wherein the biological classification ensemble model identifies the biological product type upon determination that the first indicator passes a first pass-fail based threshold value and that the second indicator passes a second pass-fail based threshold value.

Aspect 40. The biological analytics method of any one of aspects 30-38, wherein the first indicator as output by the unsupervised model is based on whether the one or more biological product types satisfies a threshold value.

Aspect 41. The biological analytics method of aspect 40, wherein the unsupervised model outputs a pass-fail determination based on the threshold value.

Aspect 42. The biological analytics method of aspect 40 or 41, wherein the threshold value is based on one or more of: a reduced Q-residual error, a Hotelling's T-squared value, a Mahalanobis distance value, or specific range values for principal component scores.

Aspect 43. The biological analytics method of any one of aspects 30-42, wherein a first biological product type of the one or more biological product types and a second biological product type of the one or more biological product types have similar Raman-based spectra.

Aspect 44. The biological analytics method of any one of aspect 30-43, wherein the second indicator as output by the supervised model is based on whether the one or more biological product types satisfies a biological product type prediction threshold value.

Aspect 45. The biological analytics method of aspect 44, wherein the supervised model outputs a pass-fail determination based on the biological product type prediction threshold value.

Aspect 46. The biological analytics method of any one of aspects 30-45, wherein the computer memory is configured to load a new biological classification ensemble model, the new biological ensemble classification model comprising an updated unsupervised model and/or an updated supervised model.

Aspect 47. The biological analytics method of any one of aspects 30-46, wherein the biological classification ensemble model configuration is implemented in an extensible markup language (XML) format.

Aspect 48. The biological analytics method of any one of aspects 30-47, wherein the biological product type is of a therapeutic product.

Aspect 49. The biological analytics method of any one of aspects 30-48, wherein the biological product type is identified by the biological classification ensemble model during manufacture of a biological product having the biological product type.

Aspect 50. The biological analytics method of any one of aspects 30-49, wherein the supervised model of the biological classification ensemble model is configured to distinguish the first biological product sample having the biological product type from a different biological product sample having a different biological product type.

Aspect 51. The biological analytics method of aspect 50, wherein the biological product type and the different biological product type each have distinct localized features within a similar Raman spectra range.

Aspect 52. The biological analytics method of any one of aspects 30-51, wherein the biological classification ensemble model is generated by a remote processor being remote to the configurable handheld biological analyzer.

Aspect 53. The biological analytics method of any one of aspects 30-52, wherein the unsupervised model is configured based on: a principal component analysis (PCA), a Euclidean distance or correlation; a neighbor-based algorithm, a K-means algorithm, Quality Threshold (QT) algorithm, a Centroid algorithm, a Ward algorithm, or a Fuzzy C-Means clustering algorithm.

Aspect 54. The biological analytics method of aspect 53, wherein the unsupervised model is a PCA model, and wherein the PCA model comprises a reduced set of principal components.

Aspect 55. The biological analytics method of any one of aspects 30-54, wherein the supervised model is trained using a partial least squares discriminant analysis (PLSDA), a linear discriminant analysis (LDA), a K-nearest neighbor (KNN) algorithm, a soft independent modeling using class analogy (SIMCA), or a logistic regression discriminant analysis (LREGDA) algorithm.

Aspect 56. The biological analytics method of aspect 55, wherein the supervised model is a PLSDA model, and wherein the PLSDA model comprises a reduced set of latent variables.

Aspect 57. The biological analytics method of any one of aspects 30-56, wherein the unsupervised model is configured based on a principal component analysis (PCA) and the supervised model is configured on a partial least squares discriminant analysis (PLSDA).

Aspect 58. The biological analytics method of any one of aspects 30-57, wherein the one or more spectral preprocessing algorithms are executed to modify at least one of: (a) training data as used to train one or both of the supervised model or the unsupervised model; or (b) production data as used to produce an output from one or both of the supervised model or the unsupervised model.

Aspect 59. A tangible, non-transitory computer-readable medium storing instructions for identification of biological products based on Raman spectroscopy using ensemble artificial intelligence (AI), that when executed by one or more processors of a configurable handheld biological analyzer cause the one or more processors of the configurable handheld biological analyzer to: load, into a first computer memory of a first configurable handheld biological analyzer having a first processor and a first scanner, a biological ensemble classification model configuration, the biological ensemble classification model configuration comprising a biological classification ensemble model comprising an unsupervised model and a supervised model, wherein the unsupervised model is trained with Raman-based spectra training data to configure the unsupervised model to output a first indicator of one or more biological product types, and wherein the supervised model is trained with Raman-based spectra training data to configure the supervised model to output a second indicator of the one or more biological product types; receive, at the first processor, a first Raman-based spectra dataset defining a first biological product sample as scanned by the first scanner; execute, by the first processor, one or more spectral preprocessing algorithms as specified by the biological ensemble classification model configuration, to reduce a spectral variance of the first Raman-based spectra dataset; and identify, with the biological classification ensemble model, a biological product type based on the first Raman-based spectra dataset.

What is claimed is:

1. The foregoing aspects of the disclosure are exemplary only and not intended to limit the scope of the disclosure.
A configurable handheld biological analyzer for identification of biological products based on Raman spectroscopy using ensemble artificial intelligence (AI), the configurable handheld biological analyzer comprising:
a first housing adapted for handheld manipulation;
a first scanner carried by the first housing;
a first processor communicatively coupled to the first scanner; and
a first computer memory communicatively coupled to the first processor,
wherein the first computer memory is configured to load a biological ensemble classification model configuration, the biological ensemble classification model configuration comprising a biological classification ensemble model comprising an unsupervised model and a supervised model,
wherein the unsupervised model is trained with Raman-based spectra training data to configure the unsupervised model to output a first indicator of one or more biological product types,
wherein the supervised model is trained with Raman-based spectra training data to configure the supervised model to output a second indicator of the one or more biological product types,
wherein the biological classification ensemble model configuration further comprises one or more spectral preprocessing algorithms, the first processor configured to execute the one or more spectral preprocessing algorithms to reduce a spectral variance of a first Raman-based spectra dataset when the first Raman-based spectra dataset is received by the first processor, and
wherein the biological classification ensemble model is configured to execute on the first processor, the first processor configured to (1) receive a first Raman-based spectra dataset defining a first biological product sample as scanned by the first scanner, and (2) identify, with the biological classification ensemble model, a biological product type of the one or more biological product types based on the first Raman-based spectra dataset.

2. The configurable handheld biological analyzer of claim 1, wherein the biological ensemble classification model configuration is electronically transferrable to a second configurable handheld biological analyzer, the second configurable handheld biological analyzer comprising:
a second housing adapted for handheld manipulation;
a second scanner coupled to the second housing;
a second processor communicatively coupled to the second scanner; and
a second computer memory communicatively coupled to the second processor,
wherein the second computer memory is configured to load the biological classification ensemble model configuration, the biological classification ensemble model configuration comprising the biological classification ensemble model, wherein the biological classification ensemble model is configured to execute on the second processor, the second processor configured to (1) receive a second Raman-based spectra dataset defining a second biological product sample as scanned by the second scanner, and (2) identify, with the biological classification ensemble model, the biological product type based on the second Raman-based spectra dataset,
wherein the second biological product sample is a new sample of the biological product type.

3. The configurable handheld biological analyzer of claim 1, wherein the spectral variance is an analyzer-to-analyzer spectral variance between the first Raman-based spectra dataset and one or more other Raman-based spectra datasets of one or more corresponding other handheld biological analyzers, each of the one or more other Raman-based spectra datasets representative of the biological product type, and
wherein the one or more spectral preprocessing algorithms are configured to mitigate the analyzer-to-analyzer spectral variance between the first Raman-based spectra dataset and the one or more other Raman-based spectra datasets.

4. The configurable handheld biological analyzer of claim 3, wherein the one or more spectral preprocessing algorithms comprises:
applying a derivative transformation to the first Raman-based spectra dataset to generate a modified Raman-based spectra dataset,
aligning the modified Raman-based spectra dataset across a Raman shift axis, and
normalizing the modified Raman-based spectra dataset across a Raman intensity axis.

5. The configurable handheld biological analyzer of claim 4, wherein the modified Raman-based spectra dataset is centered.

6. The configurable handheld biological analyzer of claim 4, wherein the derivative transformation is applied to consecutive groups of 5 to 15 Raman intensity values across the Raman shift axis.

7. The configurable handheld biological analyzer of claim 5, wherein corresponding derivatives of the consecutive groups of 5 to 15 Raman intensity values are determined across the Raman shift axis.

8. The configurable handheld biological analyzer of claim 1, wherein the unsupervised model is configured to detect variability associated with identifying the one or more biological product types.

9. The configurable handheld biological analyzer of claim 8, wherein the variability comprises instrument variability or sample lot-to-lot variability.

10. The configurable handheld biological analyzer of claim 1, wherein the biological classification ensemble model identifies the biological product type upon determination that the first indicator passes a first pass-fail based threshold value and that the second indicator passes a second pass-fail based threshold value.

11. The configurable handheld biological analyzer of claim 1, wherein the first indicator as output by the unsupervised model is based on whether the one or more biological product types satisfies a threshold value.

12. The configurable handheld biological analyzer of claim 11, wherein the unsupervised model outputs a pass-fail determination based on the threshold value.

13. The configurable handheld biological analyzer of claim 11, wherein the threshold value is based on one or more of: a reduced Q-residual error, a Hotelling's T-squared value, a Mahalanobis distance value, or specific range values for principal component scores.

14. The configurable handheld biological analyzer of claim 1, wherein a first biological product type of the one or more biological product types and a second biological product type of the one or more biological product types have similar Raman-based spectra.

15. The configurable handheld biological analyzer of claim 1, wherein the second indicator as output by the supervised model is based on whether the one or more biological product types satisfies a biological product type prediction threshold value.

16. The configurable handheld biological analyzer of claim 15, wherein the supervised model outputs a pass-fail determination based on the biological product type prediction threshold value.

17. The configurable handheld biological analyzer of claim 1, wherein the computer memory is configured to load a new biological classification ensemble model, the new biological ensemble classification model comprising an updated unsupervised model and/or an updated supervised model.

18. The configurable handheld biological analyzer of claim 1, wherein the biological classification ensemble model configuration is implemented in an extensible markup language (XML) format.

19. The configurable handheld biological analyzer of claim 1, wherein the biological product type is of a therapeutic product.

20. The configurable handheld biological analyzer of claim 1, wherein the biological product type is identified by the biological classification ensemble model during manufacture of a biological product having the biological product type.

21. The configurable handheld biological analyzer of claim 1, wherein the supervised model of the biological classification ensemble model is configured to distinguish the first biological product sample having the biological product type from a different biological product sample having a different biological product type.

22. The configurable handheld biological analyzer of claim 21 wherein the biological product type and the different biological product type each have distinct localized features within a similar Raman spectra range.

23. The configurable handheld biological analyzer of claim 1, wherein the biological classification ensemble model is generated by a remote processor being remote to the configurable handheld biological analyzer.

24. The configurable handheld biological analyzer of claim 1, wherein the unsupervised model is configured based on: a principal component analysis (PCA), a Euclidean distance or correlation; a neighbor-based algorithm, a K-means algorithm, Quality Threshold (QT) algorithm, a Centroid algorithm, a Ward algorithm, or a Fuzzy C-Means clustering algorithm.

25. The configurable handheld biological analyzer of claim 24, wherein the unsupervised model is a PCA model, and wherein the PCA model comprises a reduced set of principal components.

26. The configurable handheld biological analyzer of claim 1, wherein the supervised model is trained using a partial least squares discriminant analysis (PLSDA), a linear discriminant analysis (LDA), a K-nearest neighbor (KNN) algorithm, a soft independent modeling using class analogy (SIMCA), or a logistic regression discriminant analysis (LREGDA) algorithm.

27. The configurable handheld biological analyzer of claim 26, wherein the supervised model is a PLSDA model, and wherein the PLSDA model comprises a reduced set of latent variables.

28. The configurable handheld biological analyzer of claim 1, wherein the unsupervised model is configured based on a principal component analysis (PCA) and the supervised model is configured on a partial least squares discriminant analysis (PLSDA).

29. The configurable handheld biological analyzer of claim 1, wherein the one or more spectral preprocessing algorithms are executed to modify at least one of: (a) training data as used to train one or both of the supervised model or the unsupervised model; or (b) production data as used to produce an output from one or both of the supervised model or the unsupervised model.

30. A biological analytics method for identification of biological products based on Raman spectroscopy using ensemble artificial intelligence (AI), the biological analytics method comprising:
loading, into a first computer memory of a first configurable handheld biological analyzer having a first processor and a first scanner, a biological ensemble classification model configuration, the biological ensemble classification model configuration comprising a biological classification ensemble model comprising an unsupervised model and a supervised model,
wherein the unsupervised model is trained with Raman-based spectra training data to configure the unsupervised model to output a first indicator of one or more biological product types, and
wherein the supervised model is trained with Raman-based spectra training data to configure the supervised model to output a second indicator of the one or more biological product types;
receiving, at the first processor, a first Raman-based spectra dataset defining a first biological product sample as scanned by the first scanner;
executing, by the first processor, one or more spectral preprocessing algorithms as specified by the biological ensemble classification model configuration, to reduce a spectral variance of the first Raman-based spectra dataset; and
identifying, with the biological classification ensemble model, a biological product type based on the first Raman-based spectra dataset.

31. The biological analytics method of claim 30 further comprising:
transferring the biological ensemble classification model configuration to a second configurable handheld biological analyzer;
loading, into a second computer memory, the biological classification ensemble model configuration, the biological classification ensemble model configuration comprising the biological classification ensemble model;
receiving, by a second processor of the second configurable handheld biological analyzer, a second Raman-based spectra dataset defining a second biological product sample as scanned by the second scanner; and
identifying, by the second processor implementing the biological classification ensemble model, the biological product type based on the second Raman-based spectra dataset,
wherein the second biological product sample is a new sample of the biological product type.

32. The biological analytics method of claim 30, wherein the spectral variance is an analyzer-to-analyzer spectral variance between the first Raman-based spectra dataset and one or more other Raman-based spectra datasets of one or more corresponding other handheld biological analyzers, each of the one or more other Raman-based spectra datasets representative of the biological product type, and
wherein the one or more spectral preprocessing algorithms are configured to mitigate the analyzer-to-analyzer spectral variance between the first Raman-based spectra dataset and the one or more other Raman-based spectra datasets.

33. The biological analytics method of claim 32, wherein the one or more spectral preprocessing algorithms comprises:
applying a derivative transformation to the first Raman-based spectra dataset to generate a modified Raman-based spectra dataset,
aligning the modified Raman-based spectra dataset across a Raman shift axis, and
normalizing the modified Raman-based spectra dataset across a Raman intensity axis.

34. The biological analytics method of claim 33, wherein the modified Raman-based spectra dataset is centered.

35. The biological analytics method of claim 33, wherein the derivative transformation is applied to consecutive groups of 5 to 15 Raman intensity values across the Raman shift axis.

36. The biological analytics method of claim 35, wherein corresponding derivatives of the consecutive groups of 5 to 15 Raman intensity values are determined across the Raman shift axis.

37. The biological analytics method of claim 30, wherein the unsupervised model is configured to detect variability associated with identifying the one or more biological product types.

38. The biological analytics method of claim 37, wherein the variability comprises instrument variability or sample lot-to-lot variability.

39. The biological analytics method of claim 30, wherein the biological classification ensemble model identifies the biological product type upon determination that the first indicator passes a first pass-fail based threshold value and that the second indicator passes a second pass-fail based threshold value.

40. The biological analytics method of claim 30, wherein the first indicator as output by the unsupervised model is based on whether the one or more biological product types satisfies a threshold value.

41. The biological analytics method of claim 40, wherein the unsupervised model outputs a pass-fail determination based on the threshold value.

42. The biological analytics method of claim 40, wherein the threshold value is based on one or more of: a reduced Q-residual error, a Hotelling's T-squared value, a Mahalanobis distance value, or specific range values for principal component scores.

43. The biological analytics method of claim 30, wherein a first biological product type of the one or more biological product types and a second biological product type of the one or more biological product types have similar Raman-based spectra.

44. The biological analytics method of claim 30, wherein the second indicator as output by the supervised model is based on whether the one or more biological product types satisfies a biological product type prediction threshold value.

45. The biological analytics method of claim 44, wherein the supervised model outputs a pass-fail determination based on the biological product type prediction threshold value.

46. The biological analytics method of claim 30, wherein the computer memory is configured to load a new biological classification ensemble model, the new biological ensemble classification model comprising an updated unsupervised model and/or an updated supervised model.

47. The biological analytics method of claim 30, wherein the biological classification ensemble model configuration is implemented in an extensible markup language (XML) format.

48. The biological analytics method of claim 30, wherein the biological product type is of a therapeutic product.

49. The biological analytics method of claim 30, wherein the biological product type is identified by the biological classification ensemble model during manufacture of a biological product having the biological product type.

50. The biological analytics method of claim 30, wherein the supervised model of the biological classification ensemble model is configured to distinguish the first biological product sample having the biological product type from a different biological product sample having a different biological product type.

51. The biological analytics method of claim 50, wherein the biological product type and the different biological product type each have distinct localized features within a similar Raman spectra range.

52. The biological analytics method of claim 30, wherein the biological classification ensemble model is generated by a remote processor being remote to the configurable handheld biological analyzer.

53. The biological analytics method of claim 30, wherein the unsupervised model is configured based on: a principal component analysis (PCA), a Euclidean distance or correlation; a neighbor-based algorithm, a K-means algorithm, Quality Threshold (QT) algorithm, a Centroid algorithm, a Ward algorithm, or a Fuzzy C-Means clustering algorithm.

54. The biological analytics method of claim 53, wherein the unsupervised model is a PCA model, and wherein the PCA model comprises a reduced set of principal components.

55. The biological analytics method of claim 30, wherein the supervised model is trained using a partial least squares discriminant analysis (PLSDA), a linear discriminant analysis (LDA), a K-nearest neighbor (KNN) algorithm, a soft independent modeling using class analogy (SIMCA), or a logistic regression discriminant analysis (LREGDA) algorithm.

56. The biological analytics method of claim 55, wherein the supervised model is a PLSDA model, and wherein the PLSDA model comprises a reduced set of latent variables.

57. The biological analytics method of any one of claim 30, wherein the unsupervised model is configured based on a principal component analysis (PCA) and the supervised model is configured on a partial least squares discriminant analysis (PLSDA).

58. The biological analytics method of any one of claim 30, wherein the one or more spectral preprocessing algorithms are executed to modify at least one of: (a) training data as used to train one or both of the supervised model or the unsupervised model; or (b) production data as used to produce an output from one or both of the supervised model or the unsupervised model.

59. A tangible, non-transitory computer-readable medium storing instructions for identification of biological products based on Raman spectroscopy using ensemble artificial intelligence (AI), that when executed by one or more processors of a configurable handheld biological analyzer cause the one or more processors of the configurable handheld biological analyzer to: load, into a first computer memory of a first configurable handheld biological analyzer having a first processor and a first scanner, a biological ensemble classification model configuration, the biological ensemble classification model configuration comprising a biological classification ensemble model comprising an unsupervised model and a supervised model,
wherein the unsupervised model is trained with Raman-based spectra training data to configure the unsupervised model to output a first indicator of one or more biological product types, and
wherein the supervised model is trained with Raman-based spectra training data to configure the supervised model to output a second indicator of the one or more biological product types;
receive, at the first processor, a first Raman-based spectra dataset defining a first biological product sample as scanned by the first scanner;
execute, by the first processor, one or more spectral preprocessing algorithms as specified by the biological ensemble classification model configuration, to reduce a spectral variance of the first Raman-based spectra dataset; and
identify, with the biological classification ensemble model, a biological product type based on the first Raman-based spectra dataset.
